(19) United States
(12) Patent Application Publication
(10) Pub. No.: US 2019/0178889 A1
(43) Pub. Date: Jun. 13, 2019

(54) HIGH-THROUGHPUT SCREENING ASSAY

(71) Applicant: BellBrook Labs, Madison, WI (US)

(72) Inventors: Meera Kumar, Madison, WI (US); Tom Zielinski, Madison, WI (US); Robert G. Lowery, Madison, WI (US)

(21) Appl. No.: 15/741,460

(22) PCT Filed: Sep. 5, 2017
(86) PCT No.: PCT/US2017/050060

§ 371 Date: Jan. 2, 2018

Related U.S. Application Data
(60) Provisional application No. 62/383,566, filed on Sep. 5, 2016.

(51) Int. Cl. G01N 33/573; C07K 16/40; C12Q 1/02; C12Q 1/25; G01N 33/566; G01N 33/58; G01N 33/541

(52) U.S. Cl. CPC G01N 33/573; C07K 16/40; C12Q 1/025; C12Q 1/25; C12Y 207/07; G01N 33/582; G01N 33/541; C07K 2317/622; C07K 2317/92; G01N 33/566

(57) ABSTRACT

Methods and materials for development of high-throughput screening assays for detection of cyclic GMP (cGAMP) are provided by this invention.

### BACKGROUND OF THE INVENTION

### Field of Invention

The present invention relates to methods and materials for development of high-throughput screening assays for measuring cyclic GMP-AMP synthase (cGAS) activity and/or detecting G(2′-5′)pA(3′-5′)p (cGAMP).

### Description of Related Art

Cyclic GMP-AMP synthase (cGAS) (UniProtKB—Q8N884) is a recently discovered enzyme that acts as a DNA sensor to elicit an immune response to pathogens via activation of the stimulator of interferon genes (STING) receptor. Shortly after its discovery in 2013, aberrant activation of cGAS by self-DNA was shown to underlie debilitating and sometimes fatal autoimmune diseases, such as systemic lupus erythematosus (SLE), scleroderma, and Aicardi-Goutieres Syndrome (AGS). Knockout studies in animal models have clearly indicated that inhibiting cGAS is a promising approach for therapeutic intervention. Additionally, recent studies have shown that the cGAS-STING pathway plays a key role in the innate immune response to tumors, and stimulation of the pathway is a promising strategy being tested clinically for cancer immunotherapy.

However, there are no high-throughput screening (HTS)-compatible assay methods for measuring cGAS enzyme activity, and development of a homogenous assay presents a considerable challenge, as it requires selective detection of the cyclic dinucleotide product, cGAMP (FIG. 1), in the presence of the substrates ATP and GTP. Researchers currently are relying on radioassays coupled with thin-layer chromatography (TLC) for biochemical cGAS assays and LC-MS for detection of cGAMP in cell lysates or tissue samples. Therefore, there remains a need to develop sensors conducive to performing HTS assays for measuring cGAS activity and detecting cGAMP.

### SUMMARY OF THE INVENTION

It is against the above background that the present invention provides certain advantages and advancements over the prior art.

Although this invention disclosed herein is not limited to specific advantages or functionalities, the invention provides an antibody that specifically binds G(2′-5′)pA(3′-5′)p (cGAMP).

In one aspect of the antibodies disclosed herein, the antibody specifically binds cGAMP in the presence of excess ATP, GTP or both.

In one aspect of the antibodies disclosed herein, the cGAMP is produced in an enzymatically catalyzed reaction.

In one aspect of the antibodies disclosed herein, the antibody binds cGAMP with a K_{d} of less than 100 nM, less than 5 nM or less than 100 pM.

In one aspect of the antibodies disclosed herein, the binding is in a biological sample.

In one aspect of the antibodies disclosed herein, the biological sample is a cell extract or a tissue extract.

In one aspect of the antibodies disclosed herein, the antibody is a mouse monoclonal antibody.

In one aspect of the antibodies disclosed herein, the antibody is a single-chain variable fragment (scFv).

In one aspect of the antibodies disclosed herein, the antibody is conjugated to a Tb-chelate or an Eu-chelate.

The invention also provides an assay method for measuring cGAMP produced in an enzymatically catalyzed reaction, comprising:
(a) contacting a biological sample with the antibodies disclosed herein and a fluorescently labeled cGAMP tracer;
(b) measuring a signal; and
(c) detecting cGAMP in the sample;
thereby measuring cGAMP produced in the reaction.

In one aspect of the assay methods disclosed herein, the reaction is catalyzed by cyclic GMP-AMP synthase (cGAS).

The invention also provides an assay method for measuring cyclic GMP-AMP synthase (cGAS) activity, comprising:
(a) contacting a biological sample with the antibodies disclosed herein and a fluorescently labeled cGAMP tracer;
(b) measuring a signal; and
(c) detecting cGAMP in the sample;
thereby measuring cyclic GM P-AMP synthase (cGAS) activity.

In one aspect of the assay methods disclosed herein, the fluorescently labeled cGAMP tracer is displaced by unlabeled cGAMP in the sample.

In one aspect of the assay methods disclosed herein, the cGAMP tracer is labeled with a fluorescein, Alexa Fluor, Dylight, and/or Atto dye.

In one aspect of the assay methods disclosed herein, the signal is a time resolved Förster resonance energy transfer (TR-FRET) signal or a fluorescence polarization (FP) signal.

In one aspect of the assay methods disclosed herein, the assay method is:
(a) a competitive assay method;
(b) a homogenous assay method;
(c) an assay method having low nanomolar sensitivity;
(d) an assay method allowing for direct detection of cGAMP; and/or
(e) an assay method operable in endpoint or continuous mode.

In one aspect of the assay methods disclosed herein, the assay method is a high-throughput screening (HTS) assay method.

The invention also provides an assay kit for detecting and measuring cGAMP produced in an enzymatically catalyzed reaction, comprising the antibodies disclosed herein, ATP, GTP or both, and a fluorescently labeled cGAMP tracer.

The invention also provides an antibody pair comprising a first antibody and a second antibody, wherein:
the first antibody specifically binds G(2′-5′)pA(3′-5′)p (cGAMP) in the presence of excess ATP and GTP; and
the second antibody specifically binds:
(a) cGAMP, simultaneously with the first antibody; or
(b) a complex of the first antibody and cGAMP.

In one aspect of antibody pairs disclosed herein, the first antibody binds cGAMP with a K_{d} of less than 100 nM, less than 5 nM, or less than 100 pM; and the second antibody binds cGAMP or a complex of the first antibody and cGAMP with a K_{d} of less than 100 nM, less than 5 nM, or less than 100 pM.

In one aspect of antibody pairs disclosed herein, the first antibody and/or the second antibody comprise a single-chain variable fragment (scFv).

In one aspect of antibody pairs disclosed herein, the binding is in a biological sample.

In one aspect of antibody pairs disclosed herein, the biological sample is a cell extract or a tissue extract.

In one aspect of antibody pairs disclosed herein, the biological sample is a cell lysate.

In one aspect of antibody pairs disclosed herein, the first antibody is conjugated to a Tb-chelate or an Eu-chelate and the second antibody is conjugated to a fluorescent label.

In one aspect of antibody pairs disclosed herein, the first antibody is conjugated to a fluorescent label, and the second antibody is conjugated to a Tb-chelate or an Eu-chelate.

In one aspect of antibody pairs disclosed herein, the fluorescent label comprises a fluorescein, Alexa Fluor, Dylight, and/or Atto dye.

The invention also provides an assay method for measuring cGAMP produced in an enzymatically catalyzed reaction, comprising:
(a) contacting a biological sample with the antibody pairs disclosed herein;
(b) measuring a signal; and
(c) detecting cGAMP in the sample;
thereby measuring cGAMP produced in the reaction.

In one aspect of the assay method disclosed herein, the reaction is catalyzed by cyclic GMP-AMP synthase (cGAS).

The invention also provides an assay method for measuring cyclic GMP-AMP synthase (cGAS) activity comprising:
(a) contacting a biological sample with the antibody pairs disclosed herein;
(b) measuring a signal; and
(c) detecting cGAMP in the sample;
thereby measuring cyclic GM P-AMP synthase (cGAS) activity.

In one aspect of the assay method disclosed herein, the signal is a time-resolved Förster resonance energy transfer (TR-FRET) signal or a fluorescence polarization (FP) signal.

In one aspect of the assay method disclosed herein, the assay method is a high-throughput screening (HTS) assay method.

The invention also provides an assay kit for detecting and measuring cGAMP produced in an enzymatically catalyzed reaction, comprising the antibody pairs disclosed herein, and ATP, GTP, or both.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows a schematic structure of cGAMP.

FIG. 2 is a schematic for a cGAS enzymatic assay based on a homogenous time resolved Förster resonance energy transfer (TR-FRET) immunoassay for cGAMP. Displacement of fluorescent tracer from antibody by cGAMP disrupts energy transfer from Ab-bound lanthanide. For a fluorescence polarization (FP) assay, the antibody is unlabeled; displacement of tracer causes its polarization to decrease.

FIG. 3 is a schematic showing activation of cGAS by cytoplasmic DNA or RNA initiates activation of the innate immune response via induction of Type I interferons (IFN-I).

FIG. 4 shows specific binding of mouse antiserum to a fluorescent cGAMP tracer. Tracer was 4 nM and competitor nucleotides were 10 μM.

FIG. 5 is a schematic showing iterative co-development of cyclic guanosine monophosphate-adenosine monophosphate (cGAMP) mAbs and tracers. cGAMP mAbs, immunogen, and tracers were synthesized from aminobutyl-cGAMP and tested in matrix fashion to identify optimal pair(s) for an FP based immunoassay, as discussed in more detail in Example 1, below. cGAMP mAb6 was selected for further assay development, as discussed in more detail in Example 2, below. The same mAb was then used to develop the TR-FRET assay, which involved conjugation with different lanthanide chelates and testing with higher wavelength tracers, as discussed in more detail in Example 3, below.

FIG. 6 is a schematic showing the cGAS-cGAMP-STING pathway. Activation of cGAS by cytoplasmic DNA initiates activation of the innate immune response via induction of IFN-I which induce tumor cell specific T cell responses in cancer, but induce autoantibodies and cause extensive tissue damage in autoimmune diseases such as SLE (CTL; cytotoxic T lymphocyte).

FIG. 7 includes A) a schematic showing the central role of plasmacytoid dendritic cells (pDCs) in SLE: DNA from dying cells and neutrophil extracellular traps (NETs) drives IFN production in pDCs to initiate autoimmunity; B) a plot showing increased expression of cGAS (mRNA) in SLE patients: each symbol represents an individual subject; horizontal lines show the mean (based on analysis of peripheral blood mononuclear cells (PBMCs) from 20 heathy controls (CNT) and 51 SLE patients); C) a graph showing the correlation between cGAS mRNA expression and the IGN score in SLE patients, as determined by linear regression analysis (see An, et al., 2017, Arthritis Rheumatol 69(4):800-7).

FIG. 8 includes A) a schematic showing the FP assay principle, as discussed in more detail in Example 1, below: in the competitive fluorescence polarization (FP) assay for cGAMP, enzymatically generated cGAMP displaces a fluorescent tracer from mAb causing a decrease in its polarization; B) a set of binding curves for cGAMP mAbs and cGAMP-Atto 633 tracer: similar analyses were carried out with several tracers to select cGAMP mAb6 for further assay development; C) a set of binding curves for cGAMP mAb6 and representative cGAMP-Fluor tracers: fluors were attached to C-8 of guanine; the Atto 633 tracer was selected for further assay development; D) a set of competition curves, indicating displacement of the tracer by cGAMP and showing dependence of dynamic range on mAb concentration; E) a set of competition curves demonstrating outstanding selectivity for cGAMP vs. cGAS substrates, including ATP, GTP, and related molecules.

FIG. 9 includes A) a plot showing detection of purified, full-length cGAS: cGAS enzyme titrations show a dose-dependent assay signal, as discussed in more detail in Example 2, below; N- and C-terminal His-tagged cGAS was prepared according to Example 4 (100 μM ATP and GTP, 62.5 nM 45 bp ISD DNA, 60 min reactions); B) a plot showing the linear response of the assay of Example 2: polarization data was converted to cGAMP using a standard curve, demonstrating linearity of product formation with cGAS enzyme concentration; C) Confirmation that recombinant hcGAS was activated by ds DNA; half maximal responses of 3.5 and 5.9 nM for HSV 60 and ISD 45, respectively; D) ATP and GTP dependence: ATP and GTP were titrated separately and simultaneously; half maximal cGAS activity at 80 μM of both nucleotide; E) Dose response for cGAS inhibition by two flavonoids; F) a plot showing the results of an interference screen: the library of 1280 pharmacologically active compounds, available from Sigma (the “LOPAC library”) was used to test for compound interference with detection reagents as discussed in more detail in Example 2, below; wells contained all cGAS enzyme reaction components except the cGAS enzyme; positive controls contained 1 μM cGAMP, negative controls contained no cGAMP; G) a plot showing the results of a pilot screen with 1600 diversity compounds (Life Chemicals), as discussed in more detail in Example 2, below: cGAS was used at 10 nM, 60 min reaction; negative controls lacked dsDNA required for cGAS activation; H) an image of a coomassie blue stained SDS gel (top) and western blot (bottom) of purified 6×HIS-cGAS (Lane 1) and cGAS-6×His (Lane 2) prepared according to Example 4; Z=0.62, Z′=0.7.

FIG. 10 includes: A) a schematic showing the TR-FRET assay principle, as discussed in more detail in Example 3, below: enzymatically generated cGAMP displaces a fluorescent tracer from lanthanide-labeled mAb causing a decrease in the TR-FRET signal; B) Binding curves for cGAMP mAb 6-lanthanide conjugates and cGAMP-Atto 650 tracer: Tracer was titrated with Tb-mAb 6 held constant at 10 nM; the terbium conjugate was chosen for further assay development; C) Tracer optimization: Assay response with 3 of the cGAMP-Fluor tracers tested; the Atto-650 tracer was chosen for further assay development; D) Competition curves at different tracer concentrations: indicate displacement of tracer by cGAMP and show dependence of dynamic range on tracer concentration; E) cGAS enzyme titration using N- and C-terminal His tagged proteins prepared according to Example 4: as with the FP assay of Examples 1-2, conversion of FRET signal yielded a linear response.

FIG. 11 is a schematic of the development of high-affinity single-chain variable fragments (scFvs) with selective epitope recognition properties for cGAMP biomarker and cellular HTS assays, as discussed in more detail in Example 5, below. cGAMP antigen and cognate tracers are synthesizing by attachment to linkers at the indicated positions on cGAMP. Resulting mAbs are tested in matrix fashion with tracers to characterize affinity and epitope recognition properties. The most promising 3-4 mAbs are subjected to several rounds of affinity maturation, as scFvs, to increase affinity and epitope selectivity as required for biomarker and cellular HTS assays.

FIG. 12 is a schematic of affinity maturation using yeast display and FACS-based sorting, as discussed in more detail in Example 6, below: A) scFvs are cloned into pCTCON-T in fusion with Aga2p for yeast surface display; B) red fluorescent cGAMP analogs, with the cognate structure for each scFv of Example 5, are used at subsaturating concentrations (⅓-½ K_{d}) to label scFv variants with increased affinity. Protein A-fluorescein is used to specifically label properly folded scFvs; C) several cycles of random mutagenesis and FACS-based enrichment are used to select for 3-4 scFvs with enhanced affinity and distinct epitope recognition.

FIG. 13 is a schematic showing sandwich time-resolved Förster resonance energy transfer (S-TR-FRET) and alternative assay configurations for a cGAMP immunoassay with picomolar sensitivity, as discussed in more detail in Example 7, below: A) S-TR-FRET format; B) a sandwich ELISA with an scFv-AP fusion, which can provide increased sensitivity; C) an anti-immune complex (anti-IC) scFv, used as a secondary Ab.

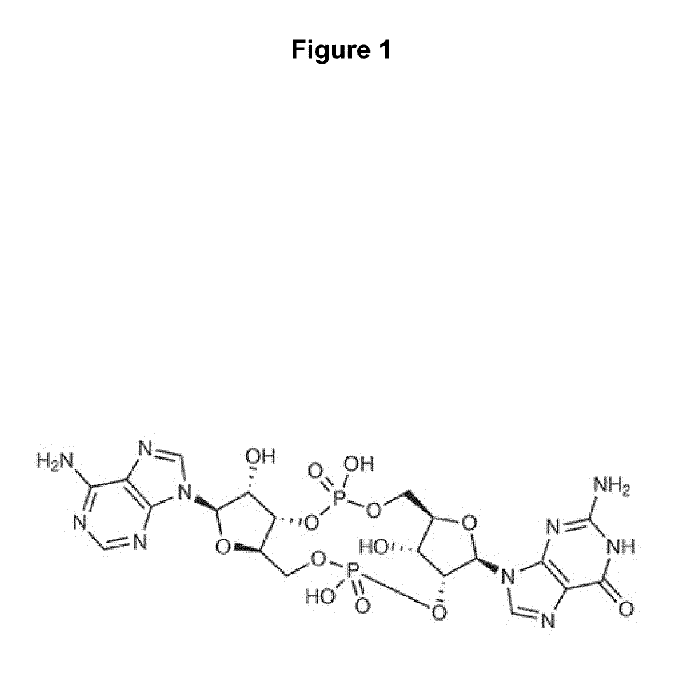

Skilled artisans will appreciate that elements in the figures are illustrated for simplicity and clarity and have not necessarily been drawn to scale. For example, the dimensions of some of the elements in the figures can be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the terms “polynucleotide,” “nucleotide,” “oligonucleotide,” and “nucleic acid” can be used interchangeably to refer to nucleic acid comprising DNA, RNA, derivatives thereof, or combinations thereof.

As used herein, the terms “homogenous assay,” “homogenous format,” and “homogenous detection” can be used to refer to detection of an analyte by a simple mix and read procedure. A homogenous assay does not require steps such as sample washing or sample separation steps. Examples of homogenous assays include time-resolved Förster resonance energy transfer (TR-FRET), fluorescence polarization (FP), fluorescence intensity (FI), and luminescence-based assays.

As used herein, the term “and/or” is utilized to describe multiple components in combination or exclusive of one another. For example, “x, y, and/or z” can refer to “x” alone, “y” alone, “z” alone, “x, y, and z,” “(x and y) or z,” “x and (y or z),” or “x or y or z.”

cGAS-cGAMP-STING Pathway

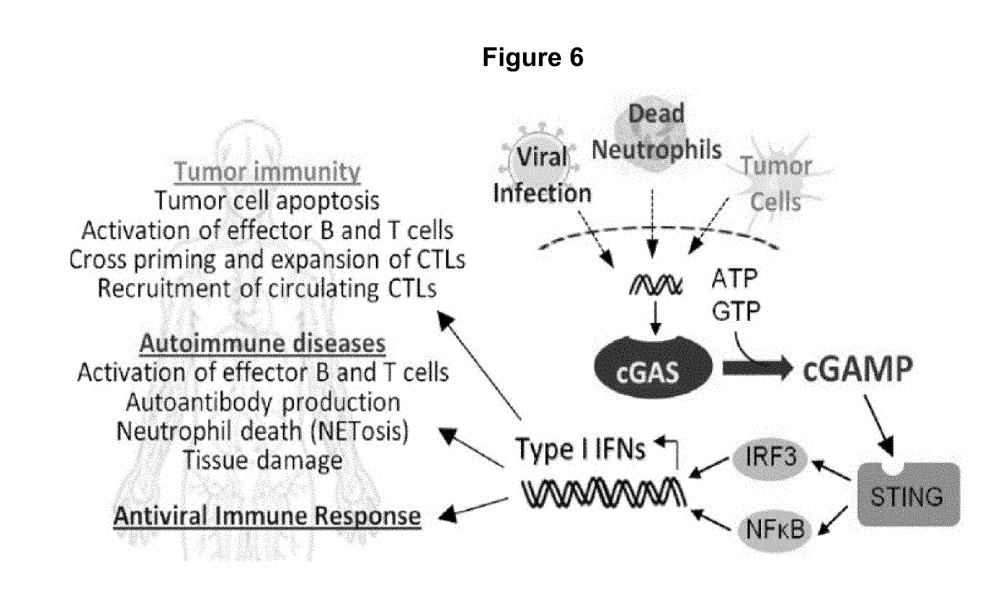

The cGAS-cGAMP-STING pathway activates the immune system in response to foreign DNA. The presence of DNA in the cytosol of eukaryotic cells is an indicator of infection or cellular damage, and it elicits a strong immune response, including type I interferon (IFN-I) induction (see FIG. 6). The STING protein was shown to mediate this response via the NF-κB and IRF3 transcription pathways in 2008, and bacterial cyclic dinucleotides were identified as STING agonists in 2011 (Burdette, et al., 2011, Nature, 478(7370):515-8; Ishikawa, et al., 2008, Nature, 455(7213):674-8). The mechanism of STING activation by DNA remained a mystery until 2013, when cyclic GAMP synthase (cGAS) was identified by two groups as the sensor for cytosolic DNA (Sun, et al., 2013, Science, 339(6121):786-91; Wu, et al., 2013, Science, 339(6121):826-30). Double stranded DNA (dsDNA) and DNA:RNA hybrids bind to a specific site on cGAS in a non-sequence-dependent manner and activate its catalytic activity, resulting in the production of a unique cyclic nucleotide G(2′-5′)pA(3′-5′)p (cGAMP) from ATP and GTP precursors (Ablasser, et al., 2013, Nature, 798(7454):380-4; Diner, et al., 2013, Cell reports, 3(5):1355-61; Kato, et al., 2013, PloS one, 8(10):376983; Mankan, et al., 2014, The EMBO journal, 33(24):2937-46). cGAMP in turn binds to the STING protein to initiate induction of the IFN-I pathway (Cai, et al., 2014, Molecular cell, 54(2):289-96). The mixed 2′-5′ and 3′-5′ phosphodiester linkages in cGAMP are not found in any known bacterial cyclic dinucleotides. Though other DNA sensors have been identified in specific types of cells, the cGAS-cGAMP-STING pathway appears to be essential for DNA-mediated immune response irrespective of cell type or DNA sequence (Cai, et al., 2014, Molecular cell, 54(2):289-96).

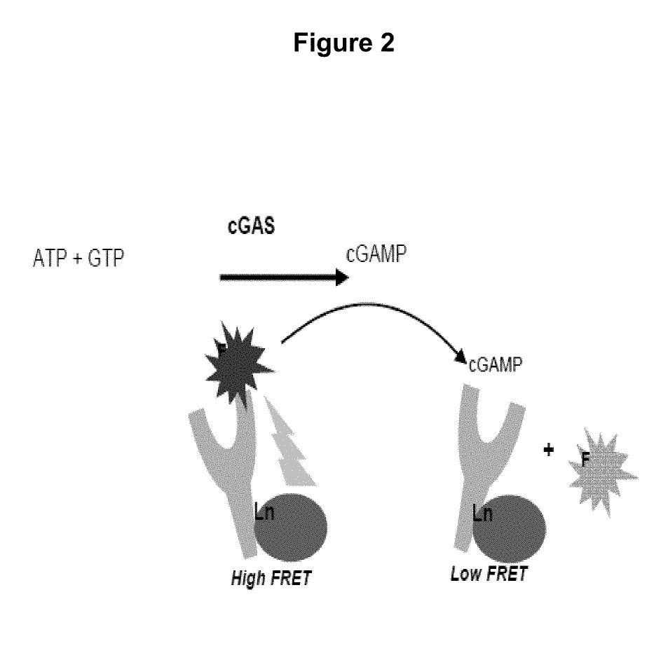

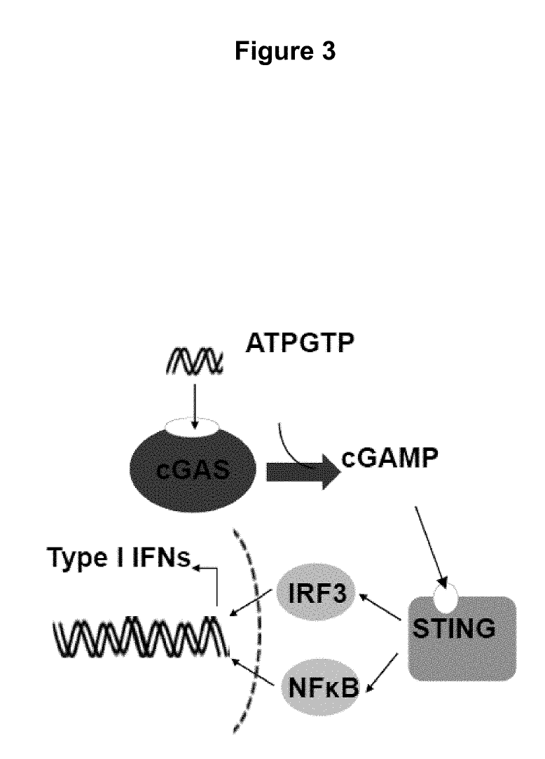

Blocking cGAS activity prevents aberrant activation of inflammatory pathways in monogenic autoimmune diseases. Inappropriate activation of the immune system by nucleic acids contributes to the pathology of a number of autoimmune diseases (FIG. 2), including Aicardi-Goutieres Syndrome (AGS), systemic lupus erythematosus (SLE), scleroderma, Sjögren's syndrome (SS), and retinal vasculopathy (Gao, et al., 2015, Proc Natl Acad Sci USA 112(42):E5699-705; Gray, et al., 2015, J Immunol. 195(5):1939-43; Mackenzie, et al., 2016, EMBO J 35(8):831-44) that cause significant pain and suffering and shorten life spans for millions of people in the U.S. alone (see Table 1). AGS, a rare neonatal encephalopathy, results in 25% mortality in early childhood, with very few patients surviving into their teens. SLE, a much more common disease, is not usually directly fatal, but it significantly increases the risk of cardiovascular diseases, and 20% of patients die within 15 years of diagnosis. Treatment of these diseases relies heavily on nonspecific immunosuppressive agents which have serious, deleterious side effects. Elevated expression of IFN-stimulated genes (ISGs) is a hallmark of nucleic acid-driven autoimmune diseases, and a number of drugs that target IFN are currently in the clinic; e.g., mAbs that bind IFNα or IFNAR, the IFN-I receptor (Weidenbusch, et al., 2017, Clin Sci (Lond) 131(8): 625-634). Multiple lines of evidence have shown that activation of the cGAS-cGAMP-STING pathway by cytoplasmic nucleic acids DNA is one of the key triggers for the pathogenic IFN responses (Cai, et al., 2014, Mol cell 54(2):289-96). Small molecule cGAS inhibitors would potentially have significant advantages in terms of cost, dosing, and pharmacodynamics over the anti-IFN biologics currently in the clinic.

TABLE 1
Autoimmune disease triggered by cGAS-
STING responses to nucleic acids

| Disease | Primary Tissue | Prevalence in U.S. |
|---|---|---|
| SLE | Many | ~1.5M |
| Scleroderma | Skin | ~150,000 |
| AGS | Brain, skin | Very rare |
| SS | Many | ~1.5M |

Mouse models have provided compelling evidence for the involvement of the cGAS-cGAMP-STING pathway—and specifically cGAS—in the pathogenesis of monogenic autoimmune diseases and, by extension, at least some types of SLE. 85% of AGS patients carry mutations in the DNA exonuclease Trex1 or RNase H2, which degrade dsDNA and DNA:RNA hybrids, respectively; knocking out and/or introducing AGS mutations to these nucleases causes lethal autoimmune disease in mice (Mackenzie, et al., 2016, EMBO J 35(8):831-44; Rice, et al., 2015, J Clin. Immunol. 35(3):235-43). Genetic ablation of cGAS or STING in nuclease-deficient mice protects against lethality and eliminates key autoimmune phenotypes, including ISG induction, autoantibody production, and T-cell activation (Gao, et al., 2015, Proc Natl Acad Sci USA 112(42):E5699-705; Gray, et al., 2015, J Immunol 195(5):1939-43; Mackenzie, et al., 2016, EMBO J 35(8):831-44; Yang, et al., 2007, Cell 131(5):873-86; Pokatayev, et al., 2016, J Exp Med 213(3):329-36). Similar results were observed when cGAS was eliminated in mice lacking DNase II, a lysosomal endonuclease that clears DNA from dead cells (Gao, et al., 2015, Proc Natl Acad Sci USA 112(42):E5699-705). Mutations that impair the function of RNase H2, Trex1, and other nucleic acid modifying enzymes are found less frequently in SLE (Mackenzie, et al., 2016, EMBO J 35(8):831-44) and in familiar chilblain lupus (Lee-Kirsch, et al., 2007, J Mol Med 85(5):531-7).

Furthermore, elevated levels of cGAMP were detected in Trex1 deficient mice, and knocking out cGAS prevented its accumulation (Gao, et al., 2016, Proc Nat Acad Sci USA 112(42):E5699-705). Similar results were observed when cGAS was eliminated in mice lacking DNasell, a lysosomal endonuclease that clears DNA from dead cells (Gao, et al., 2016, Proc Nat Acad Sci USA 112(42):E5699-705). RNA:DNA hybrids, which can be generated during aberrant DNA replication, can also induce a cGAS-cGAMP-STING dependent IFN-I response in cells, and activation of cGAS by RNA/DNA hybrids was demonstrated in in vitro biochemical assays (Mankan, et al., 2014, The EMBO journal, 33(24):2937-46). Mutations that impair the function of RNase H2, the major enzyme responsible for clearing DNA:RNA hybrids, are the predominant cause of AGS and are found less frequently in SLE (Mackenzie, et al., 2016, The EMBO journal, 35(8):831-44). Very recently, two groups showed that mice lacking functional RNaseH2 show strong ISG transcript upregulation, and elimination of cGAS—or STING—in the RNaseH2 deficient mice rescued the inflammatory phenotypes (Mackenzie, et al., 2016, EMBO J, 35(8):831-44; Yang, et al., 2007, Cell, 131(5):873-86; Pokatayev, et al., 2016, J Exp Med, 213(3): 329-36).

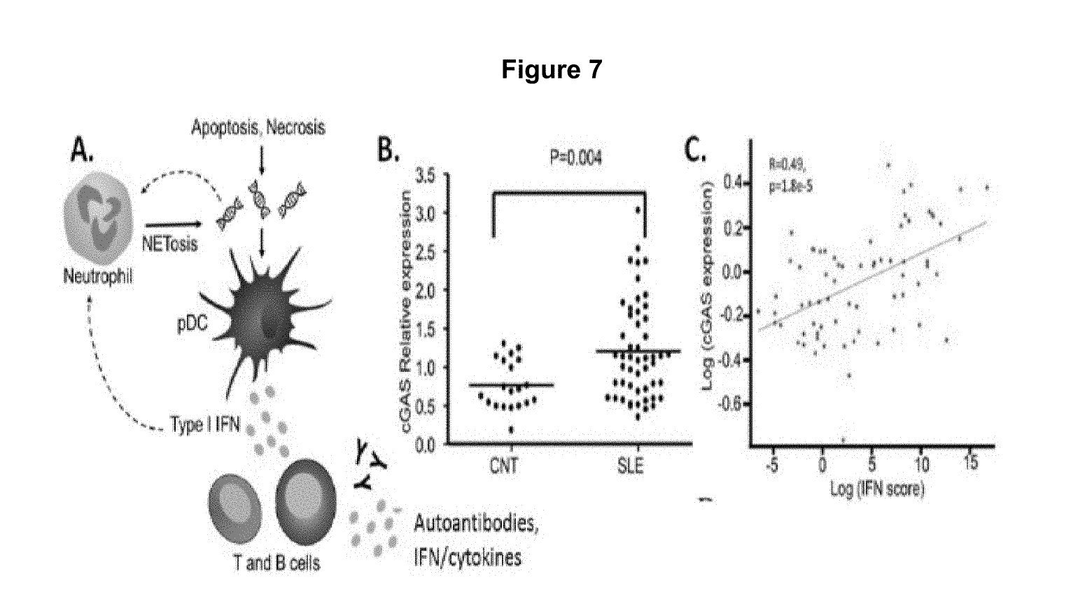

The cGAS/STING pathway drives IFN production in pDCs, and it is activated in SLE patients. The case for targeting cGAS in idiopathic SLE is rapidly building. IFN-I are strongly implicated in the pathogenesis of SLE (Elkon and Wedeman, 2012, Curr Opin Rheumatol 24(5):499-505), and approximately two thirds of SLE patients have a blood interferon (IFN) signature (Baechler, et al., 2003, Proc Nat Acad Sci USA 100(5):2610-5). Plasmacytoid dendritic cells (pDCs) are the most prolific producers of IFN-I, and their continuous stimulation is a major driver of SLE progression (Ronnblom, et al., 2003, Autoimmunity 36(8):463-72) (FIG. 7A); drugs that target pDSs in SLE have recently advanced into the clinic (21). Recently, the cGAS-cGAMP-STING pathway was shown to be required for induction of an IFN-I response by cytosolic DNA in pDCs; the response was independent of TLR9, the other major DNA sensor (Bode, et al., 2016, Eur J Immunol 46(7):1615-21). Additionally, cGAS/STING recently was shown to drive IFN-I induction in response to oxidized mitochondrial DNA from neutrophil extracellular traps (NETs) (Lood, et al., 2016, Nat Med 22(2):146-53), complexes of histones, DNA, and proteases that contribute to pathogenesis in SLE and other autoimmune diseases (FIG. 7A). Given the pivotal role of cGAS-STING in generation of IFN-I and the central role played by IFN-I in the pathogenesis of SLE, cGAS expression and cGAMP production in SLE patients was examined (An, et al., 2017, Arthritis Rheumatol 69(4):800-7). The study showed that there was an increase in cGAS transcript expression in ˜⅓ of SLE patients (FIG. 7), and that ˜15% of patients had detectable cGAMP (1˜50 nM) in peripheral blood cells (PBMCs) (FIG. 7B). Of considerable clinical significance, cGAMP+ patients had significantly higher disease activity compared to patients without increased cGAMP (FIG. 7C). Consistent with these results, it has also been reported that mRNA for TBK1, a kinase in the cGAS/STING pathway, is significantly increased in SLE patients, especially in monocytes (Hasan, et al., 2015, J Immunol 195(10):4573-7). Taken together, these studies make a compelling case for blocking cGAMP production with a small molecule inhibitor as a therapeutic intervention for SLE, AGS, and other autoimmune diseases.

Stimulation of the cGAS-cGAMP-STING pathway is also a promising approach for cancer immunotherapy (Lemos, et al., 2014, Expert Rev Clin Immunol 11(1):155-65). Immunotherapy approaches such as immune checkpoint blockade are transforming oncology, however many patients do not respond to existing agents, and alternatives are needed. In vitro and in vivo studies have shown that activation of the cGAS-STING pathway in pDCs by DNA from tumor cells and/or dead host cells is an important mechanism for initiating an antitumor T cell response in the tumor microenvironment (Ohkuri, et al., 2014, Cancer Immunol Res 2(12):1199-208; Woo, et al., 2014, Immunity 41(5):830-42) (FIG. 6). Pharmacologic targeting of the pathway is in the nascent stages, but it has already shown compelling effects. Direct injection of cGAMP or analogs thereof has potent, IFN-dependent anti-tumor effects in mouse models for glioma (Ohkuri, et al., 2014, Cancer Immunol Res 2(12):1199-208), melanoma (Corrales, et al., 2015, Cell Rep 11(7):1018-30), colon cancer (Corrales, et al., 2015, Cell Rep 11(7):1018-30; Li, 2016, et al., Sci Rep 6:19049), acute myeloid leukemia (AML) (Curran, et al., 2016, Cell Rep 15(11):2357-66), breast cancer (Corrales, et al., 2015, Cell Rep 11(7):1018-30), lung cancer (Downey, et al., 2014, PloS One 9(6):e99988), and squamous cell carcinoma (Ohkuri, et al., 2017, 66(6):705-16). Injection of cGAMP analogs generates systemic, long-term adaptive immune responses resulting in rejection of secondary non-injected tumors and distant metastases and resistance to autologous tumor re-challenge (Corrales, et al., 2015, Cell Rep 11(7):1018-30). Intratumoral injection of STING agonists is currently being tested in patients with advanced or metastatic solid tumors or lymphomas (clinicaltrials.gov, NCT02675439). However, the impact of the current STING agonists as drugs is severely limited by their pharmacokinetic liabilities, i.e., the requirement for intratumoral injection. Activating cGAS with a small molecule drug may be a more effective strategy for stimulating the STING pathway, as it would allow oral dosing and systemic distribution. In addition, there are intrinsic pharmacodynamic advantages in targeting the signal-generating enzyme rather than the receptor; for example but not limited to, significantly less drug is required to target the signal-generating enzyme.

cGAMP Detection Methods

Simple, economical methods for detecting cGAMP will have a profound impact on efforts to elucidate the roles of the cGAS/STING pathway in immunity and to target it therapeutically. Despite rapid progress since their discovery in 2013, there are major gaps in the understanding of how cGAS and cGAMP function, both at the cellular and biochemical level. Moreover, there are very few tools for discovering and developing drugs that target the pathway, especially methods that are compatible with an automated HTS approach. Currently, detection of cGAMP in cell or tissue samples requires extraction and analysis by LC/MS (An, et al., 2017, Arthritis Rheumatol 69(4):800-7; Gray, et al., 2015, J Immunol 195(5):1939-43), and there are no methods for direct detection of cGAMP in cell lysates. The only reported method for measuring cGAS enzyme activity in vitro is the semi-quantitative determination of radioactive cGAMP formation from radiolabeled ATP and GTP precursors (Ablasser A, et al., 2013, Nature 498(7454):380-4; Diner, et al., 2013, Cell Rep 3(5):1355-61; Kranzusch, et al., 2013, Cell Rep 3(5):1362-8). Aside from the undesirability of radioassays, this method requires separation by thin layer chromatography (TLC), and thus is not amenable to HTS.

The ability to readily detect cGAMP without chromatographic separation and/or radioactive labeling would greatly accelerate basic research and drug discovery targeting the pathway. Development of a selective cGAMP antibody is an important milestone. The homogenous, fluorescent assays described herein for detecting cGAMP can have a profound impact on basic research, drug discovery, and translational studies targeting STING/cGAS for autoimmune diseases and cancer immunotherapy.

cGAS activity assays will enable basic enzymological research and screening for small molecule modulators. The cumbersome and semi-quantitative nature of the radioassays and LC/MS methods used to measure cGAS activity have hindered meaningful enzymological studies. Much of the critical information needed for understanding the physiological roles of cGAS and pursuing a targeted discovery program, such as the kinetics of activation by nucleic acids of Kₘ values for ATP and GTP, is either non-quantitative or completely lacking. For example, the k_{cat} value measured and provided herein by the instant inventors was the first quantitative determination of cGAS. The biochemical HTS assay for cGAS disclosed herein enables quantitative cGAS enzymological studies as well as HTS campaigns to discover first-in-class immunotherapy drugs for devastating autoimmune diseases and cancer.

Assays for detection of cGAMP in extracted cell and tissue samples will allow investigation of cGAMP as a biomarker for disease status and drug action. There is compelling evidence that pharmacological modulation of cGAMP could be used to treat serious autoimmune diseases and to invoke an anti-tumor T cell response. Assays for detecting cGAMP in cell and tissue samples from animals and humans would provide a simple, direct way to monitor the action of lead molecules and/or experimental drugs that target cGAS, and eventually for identification of responders in clinical studies, e.g., SLE patients with high levels of cGAMP in PBMCs as candidates for cGAS inhibitors (An, et al., 2017, Arthritis Rheumatol 69(4):800-7).

Methods for direct, in-well detection of cGAMP in cell lysates will enable basic cellular studies and cellular HTS assays. As the primary signaling molecule that pDC cells use to initiate responses to viral and tumor cell DNA (Chen, et al., 2016, Nat Immunol 17(10):1142-9), upstream of both T and B cell activation, cGAMP plays a fundamental roles in human immunity. Yet very little is known about the dynamics of cGAMP levels inside and outside the cell and how they impact STING activation. cGAS is cytosolic, and it is known that cGAMP is transferred between cells through gap junctions (Chen, et al., 2016, Nat Immunol 17(10):1142-9). However, exogenously added cGAMP activates the STING pathway, both in cultured cells and in animals, and the only known cGAMP phosphodiesterase, ENPP1, functions extracellularly (Chen, et al., 2016, Nat Immunol 17(10):1142-9), suggesting that there may be specific transporters. Development of simple assays for cGAMP detection directly in culture media and cell lysates will enable detailed, quantitative studies of how the unique immunomodulator controls STING pathway activation. From a drug discovery perspective, cellular cGAS assays disclosed herein will be used to test the cellular activity of cGAS modulators identified in biochemical screens and to allow cellular screening for compounds that activate or inhibit cGAS indirectly, e.g., by modulating the uptake or intracellular production of stimulatory DNA.

Assays for the cyclic nucleotides that serve as secondary messengers for G protein-coupled receptors (GPCRs), cAMP and cGMP, are a useful comparison. Since their introduction in the early 2000's, fluorescence-based assays for cAMP have become an invaluable tool for elucidating GPCR function, and they are still the predominant HTS method used for discovery of new GPCR modulating drugs (Norskov-Lauritsen, et al., 2014, Int J Mol Sci 15(2):2554-72). Development of cGAMP assays could make a similar impact, but development is more challenging: whereas cAMP acts at micromolar concentrations, cGAMP binds STING with low nanomolar affinity, thus its detection in cells requires greater sensitivity.

The disclosure provided herein includes the development and validation of cGAS enzymatic assays, establishing key feasibility for the development of highly specific cGAMP antibodies and fluorescent tracers. The disclosure further includes the optimization of assay reagents and detection formats to detect cGAMP in cell lysates and tissue samples.

Development of cGAMP immunoassays with FP and TR-FRET readouts enables mix-and-read cGAS enzyme assays with the sensitivity and robustness required for automated HTS platforms. Applicants have previously developed the use of homogenous immunodetection of nucleotides for HTS enzyme assays (Lowery, et al., 2006, Expert Opin Ther Targets, 10(1):179-90). Applicants' Transcreener assays for ADP, GDP, UDP, and AMP/GMP have been broadly used in tens of millions of wells of pharma, biotech, and academic HTS for diverse enzyme targets including kinases, ATPases, GTPases, and glyscosyltransferases (Huss, et al., 2007, J. Biomol Screen, 12(4):578-84; Reichman, et al., 2015, J. Biomol Screen, 20(10):1294-9; Liu, et al., 2007, Assay Drug Dev Techol., 5(2):225-35). See, also, U.S. Pat. Nos. 7,332,278, 7,355,010, 7,847,066, 7,378,505, and 8,088,897, which have been incorporated herein by reference in their entirety. Antibodies that selectively recognize nucleotides that differ by as little as a single phosphate (Lowery, et al., 2006, Expert Opin Ther Targets, 10(1):179-90; Klenman-Leyer, et al., 2009, Assay Drug Dev Technol., 7(1):56-67) are the core of the technology disclosed herein.

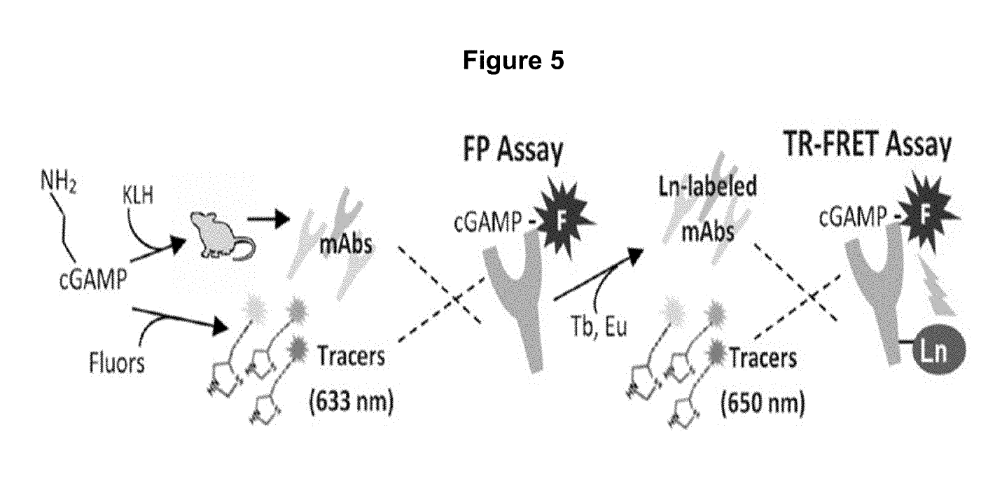

To enable HTS efforts targeting cGAS, competitive immunoassays for cGAMP have been developed with fluorescence polarization (FP) and TR-FRET signals (FIG. 2). The two major components of the HTS assay disclosed herein are the anti-cGAMP mAb (which selectively bind cGAMP in the presence of excess ATP and GTP) and the fluorescent tracer (which is displaced by cGAMP). The affinity and specificity of the mAb-cGAMP interaction, and the resultant changes in tracer fluorescence properties define the overall performance of the assay, including sensitivity, dynamic range, and signal to noise. An iterative co-development approach was used for the antibody and tracer molecules (FIG. 5). Competitive FP immunoassays are the simplest format, as they require only a tracer and an unmodified antibody; TR-FRET assays require attachment of a lanthanide chelate to the antibody for generating FRET to the bound tracer. Therefore, an FP assay was developed first, and the most promising mAb(s) were then used for development of the TR-FRET format (see Example 1).

In some embodiments, monoclonal antibodies to cGAMP were generated in mice. See Example 1. However, in other embodiments, polyclonal antibodies to cGAMP are generated in rabbits. The polyclonal antibodies were purified to obtain cGAMP-specific antibodies prior to labeling with lanthanides. In some embodiments, polyclonal antibodies were used for FP assays.

In some embodiments, human cGAS was expressed in E. coli to produce a functionally pure, active enzyme. In some embodiments, solubility of cGAS was optimized and/or cGAS was crystallized. Crystallization parameters such as culture temperature, inducer concentration, and E. coli host strain were modified for crystallization. In some embodiments, detection of cGAMP produced by cGAS was optimized using competitive immunoassays. Next, pilot screens were performed with a 1280 compound drug library and a 20K diversity library. See Example 2. In some embodiments, agents were tested to reduce non-specific binding to an antibody or tracer; e.g., non-ionic detergents, carrier proteins (bovine serum albumin (BSA) and bovine gamma globulin (BGG)). In some embodiments, cGAS was further purified using gradient elution from a cation exchange resin such as SP-Sepharose.

In further embodiments, cGAMP detection methods for cellular cGAS HTS assays and for translational studies with animal models were developed. Assay methods were optimized for detection of cGAMP in cell lysates and tissue samples as a marker for activation of the cGAS-cGAMP-STING pathway. Detection of cGAMP in biological samples is currently dependent on LC-MS. Development of a simple, homogenous assay can have very broad impact, not only on HTS efforts targeting cGAS or upstream targets (e.g., DNA uptake machinery), but also for monitoring cGAS activation status in tissue samples from animal models or patients. The lysate/tissue assays require a cGAMP antibody with negligible cross-reactivity to any other cellular nucleotides. Profiling the selectivity of the monoclonal antibodies identified in Example 1 against diverse nucleotides provide information needed to design alternative immunogens (e.g., conjugation to different sites on cGAMP), if necessary, to eliminate off-target binding (Staeben, et al., 2010, Assay Drug Dev Techol., 8(3):344-55; Klenman-Leyer, et al., 2009, Assay Drug Dev Technol., 7(1):56-67). Furthermore, FP and TR-FRET based competitive immunoassays for cyclic AMP (the mononucleotide) are widely used as cellular HTS assays for GPCR activation (Degorce, et al., 2009, Curr Chem Genomics, 3:22-32; Staeben, et al., 2010, Assay Drug Dev Technol., 8(3):344-55).

In some embodiments, the potential for compound interference with the cGAMP FP and TR-FRET assays was tested using the 1280 compound LOPAC library of pharmacologically active compounds (Sigma), which includes many scaffolds found in larger screening libraries. Assay robustness was assessed using a larger 20K set of compounds in an orthogonally pooled library from Lankenau Institute of Medical Research (LIMR) (Donover, et al., 2013, Comb Chem High Throughput Screen, 16(3):180-8). LIMR's library has been filtered for adherence to Lipinski's rule of five and lack of reactive groups. Principal component analysis indicated that ˜30%-50% of the collection represents unique chemical assemblies that are not present in the NIH-MLCPN library or commercial collections; it has been used to generate tractable scaffold series for diverse target types (Cheng, et al., 2013, J. Lab Autom., 19(3):297-303; Malecka, et al., 2014, ACS Chem Biol., 9(7):1603-12; Thompson, et al., 2010, J. Biomol Screen, 15(9):1107-15; Fera, et al., 2012, Chem Biol., 19(4):518-28).

In some aspects, the interference pre-screen was performed for both the FP and TR-FRET assays. These reactions mimic completed cGAS reactions; i.e., 10% conversion of ATP/GTP to cGAMP, but lack enzyme. Thus, they allow identification of compounds that cause an increase or decrease in the expected signal because of interference with the detection reagents. In some embodiments, following the interference pre-screen, live screens were performed with the LOPAC library or a larger diversity set using a cGAMP assay that provides the best performance, including resistance to interference. In some embodiments, all compounds were provided pre-dispensed in assay-ready plates at 10 μM assay concentration, n=1. In some embodiments, hits were confirmed with dose response measurements using the orthogonal Transcreener cGAMP assay method.

HTS enzyme assays were generally run under initial velocity conditions; i.e., less than 20% conversion of substrates to products, therefore the cGAS assay method disclosed herein requires an antibody that specifically binds cGAMP in the presence of an excess of the substrates, ATP and GTP. In some embodiments, an antibody with 100-fold selectivity fulfills this requirement and produces a very good signal (Staeben, et al., 2010, Assay Drug Dev Technol., 8(3):344-55; Klenman-Leyer, et al., 2009, Assay Drug Dev Technol., 7(1):56-67).

Sensitivity requirements are determined largely by the kinetic properties of the target enzyme. Most biochemical screens are performed with substrates at their Kₘ concentrations to insure detection of competitive inhibitors. So, for measuring enzyme initial velocity, an assay must be capable of robust detection of reaction products at concentrations several-fold below the substrate Kₘ. Though the kinetic parameters of the cGAS enzyme have not yet been reported, the target disclosed herein was a robust detection of 500 nM cGAMP. This sensitivity can allow for the use of ATP and GTP concentrations as low as 5 μM, which is likely to be well below their Kₘ values given their high micromolar concentrations in the cell.

In some embodiments, mAb/tracer pair(s) that enable detection of 500 nM cGAMP in the presence of 5 μM ATP and GTP with a Z′ greater than 0.6 and with signal stability of at least 6 hours were used. In some embodiments, mAb/tracer pair(s) that enable detection of cGAMP over a range of 0.1 μM to 50 μM were used. In some embodiments, antibodies with at least 100-fold selectivity for cGAMP vs. ATP and GTP were used in the assays disclosed herein. In some embodiments, demonstration of a linear response in cGAMP formation to cGAS concentration, time, and ATP and GTP (at concentrations below Kₘ) was achieved. In some embodiments, initial velocity cGAS activity (≤10% consumption of substrates) was detected with a Z′ value greater than 0.6 using ATP and GTP at their Kₘ concentrations. In some aspects, less than 0.5% interference in the pre-screen and Z′ values greater than 0.5 in live pilot screens were observed. In some embodiments, mAb/tracer pair(s) that produce a signal of more than 100 mP using less than 10 nM cGAS under initial velocity conditions were used. Such a signal enables screening of 1,000,000 wells with 12 mg of enzyme. In some aspects, Z′ values of more than 0.7 and/or Z values of more than 0.6, and interference levels of less than 0.4% were observed.

The assays described herein surprisingly and unexpectedly comprise the following advantages: (1) far red FP and TR-FRET signals—sensitive and resistant to compound interference, which are widely used in HTS assays; (2) homogenous assays—mix and read format is highly preferred for HTS because it simplifies automation; (3) low nanomolar sensitivity—enables cost effective screening of cGAS under initial velocity conditions; (4) direct detection—assay does not rely on coupling enzymes, which are prone to interference; and (5) usable in endpoint or continuous mode—provides flexibility for experimental protocols and applications. The novel assay disclosed herein eliminates the technical hurdle preventing screening for cGAS modulators, thereby opens up investigation of promising new therapeutic approaches for debilitating and fatal autoimmune diseases and for cancer immunotherapy. Development of cGAMP antibodies is novel and could have broad utility for drug discovery and diagnostic applications targeting the cGAS/STING pathway.

Detection of endogenous cGAMP in biological samples requires a higher sensitivity than does detection of purified cGAS (see, e.g., Table 3, below). In some embodiments, biomarker samples for LC/MS were prepared by isolating large numbers of cells (10⁶-10⁷) and resuspending in volumes of a few microliters, resulting in minimal dilution of cellular metabolites. Based on the limited information available in published studies (An, et al., 2017, Arthritis Rheumatol 69(4):800-7; Gao et al., 2015, Proc Natl Acad Sci USA 112(42):E5699-705), cGAMP concentrations in samples prepared this way are in the 1-100 nM nanomolar range.

Detection of cGAMP for cellular HTS assays requires even greater sensitivity, as they are performed by lysing cells directly in the wells where cells are cultured, and thus rely on far fewer cells (10⁴-10⁵ depending on cell type and plate density), with a dilution factor of approximately 100-fold (Fujioka, et al. Dynamics of the Ras/ERK MAPK cascade as monitored by fluorescent probes. J Biol Chem. 2006; 281(13):8917-26).

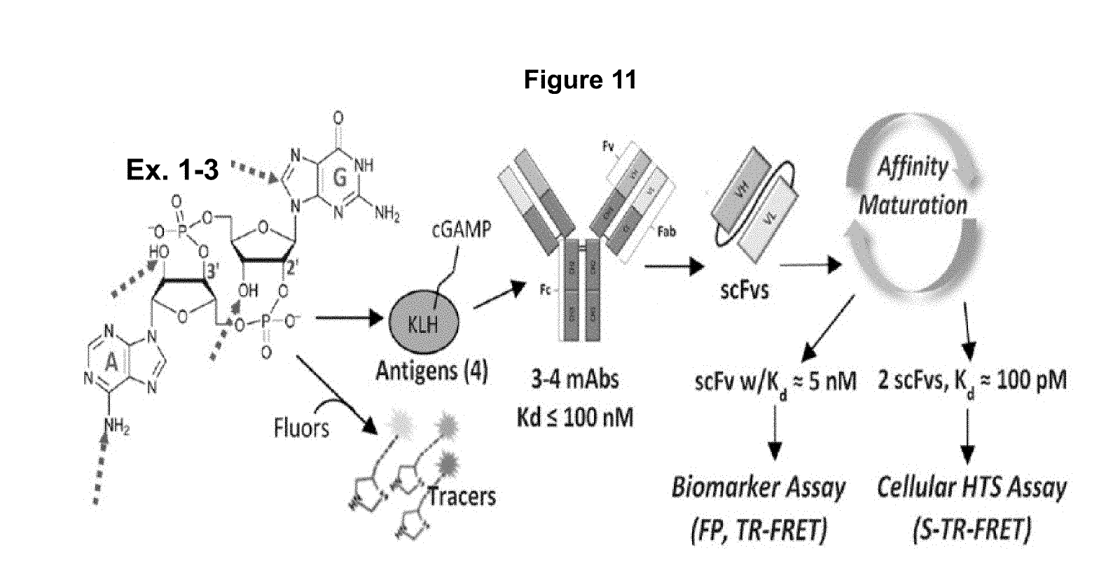

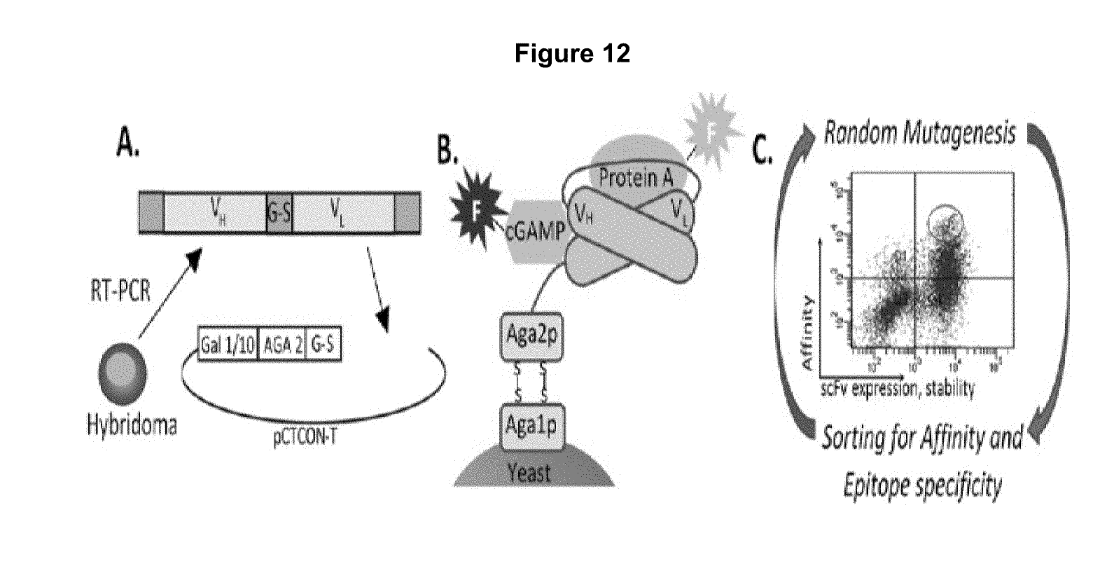

In some aspects, cGAMP detection in cell lysates requires an assay with a useful range of 20 pM to 2 nM. In some embodiments, achieving these levels of sensitivity will require antibodies with cGAMP affinities in the lower end of the detection range, e.g., a K_{d} of 5 nM for the biomarker assay and 100 pM for the cellular HTS assay. Though antibodies against small haptens tend to have lower affinities compared with antibodies for proteins, obtaining a cGAMP antibody with low nanomolar affinity is quite reasonable: for example, Applicants' Transcreener ADP antibody—a native mouse monoclonal—binds with an affinity of approximately 12 nM (Kleman-Leyer, et al., 2009, Assay Drug Dev Technol 7(1):56-67). Developing a cGAMP antibody with affinity in the picomolar range is likely beyond the capacity in vivo affinity maturation (Boder, et al., 2009, Proc Nat Acad Sci USA 97(20):10701-5). In some embodiments, in vitro evolution was used to achieve a necessary binding affinity. In some embodiments, successive rounds of random mutagenesis of V_{H} and V_{L} domains was performed, and FACS was used to enrich for desirable properties of scFvs displayed on yeast (see FIGS. 11 and 12). Such an approach has been used to achieve affinity enhancements of more than 1000-fold, well into the picomolar range, for small molecular weight antigens (Boder, et al., 2009, Proc Nat Acad Sci USA 97(20):10701-5; Boder, et al., 2012, Arch Biochem Biophys 526(2):99-106; Orcutt, et al., 2011, Nucl Med Biol 38(2):223-33; Siegel, et al., 2008, Clin Chem 54(6):1008-17) (see Table 2, below).

TABLE 2
Examples of affinity maturation of scFvs against haptens

| Hapten | Display | K_{d} (enhancement) |
|---|---|---|
| Cortisol | Phage/E. coli | 9.1 × 10−10 (7.9-fold) |
| Fluorescein | Yeast | 2.7 × 10−13 (2600-fold) |
| Tacrolimus | Yeast | 3.8 × 10−11 (15-fold) |
| Estradiol | Phage/E. coli | 7.7 × 10−11 (151-fold) |
| DOTA | Yeast | 8.2 × 10−12 (1000-fold) |

Additional key factors impacting sensitivity include the assay configuration and signaling mechanism used for detection. Competitive displacement assays (see Examples 1-3) generally have a lower limit of detection of approximately 0.5-1 nM, because they rely on a negative signal and cannot be configured for signal amplification. In contrast, dual antibody assays, whether solid phase (e.g., ELISA), or proximity based (e.g., TR-FRET) provide greater sensitivity, dynamic range and signal: background, and are often used to detect analytes in the low picomolar range (Arola, et al., 2016, Anal Chem 88(4):2446-52; Arola, et al., 2017, Toxins 9(4):145; Enomoto, et al., 2002, J Pharm Biomed Anal 28(1):73-9). While it has been assumed for many years that the simultaneous binding to two antibodies to a small molecule such as cGAMP is sterically prohibited, recent studies have challenged this limitation with the finding that separating small haptens (histamine, MW=111 and homovanillic acid, MW=182) by as few as five carbons can be sufficient to allow simultaneous binding of two antibodies (Quinton, et al., 2010, Anal Chem 82(6):2536-40). Moreover, there are examples of ELISAs developed for analytes of similar size as cGAMP. Native polyclonal antibodies against two fragments of imantinib (MW=493.6) were used to develop a sandwich ELISA with a working range of 130 pM to 16 nM (Saita, et al., 2017, Anal Chim Acta 969:72-8). A sandwich ELISA was developed for tacrolimus (MW=804) by using mAbs raised against the intact molecule linked to carrier protein via two different positions (Wei, et al., 2014, Clin Chem 60(4):621-30). The specificity of both these ELISAs was significantly better than competitive assays with single antibodies. In some aspects, cGAMP, with a molecular weight of 718 and the equivalent of more than 10 carbons between the adenine and guanine moieties, is a good candidate for development of a sandwich ELISA. In some embodiments, in vitro evolution is utilized to enhance the epitope recognition properties of the candidate mAbs (as scFvs), rather than relying on native antibodies.

In some embodiments, mAbs to cGAMP are generated using structurally distinct antigens. In some embodiments, at least one pair of antibodies, each with Kd≤100 nM, are generated. In some embodiments, at least one pair of antibodies, each exhibiting some differences in epitope recognition properties, are generated. In certain embodiments, mAbs are produced using antigens that completely lack adenine or guanine rings.

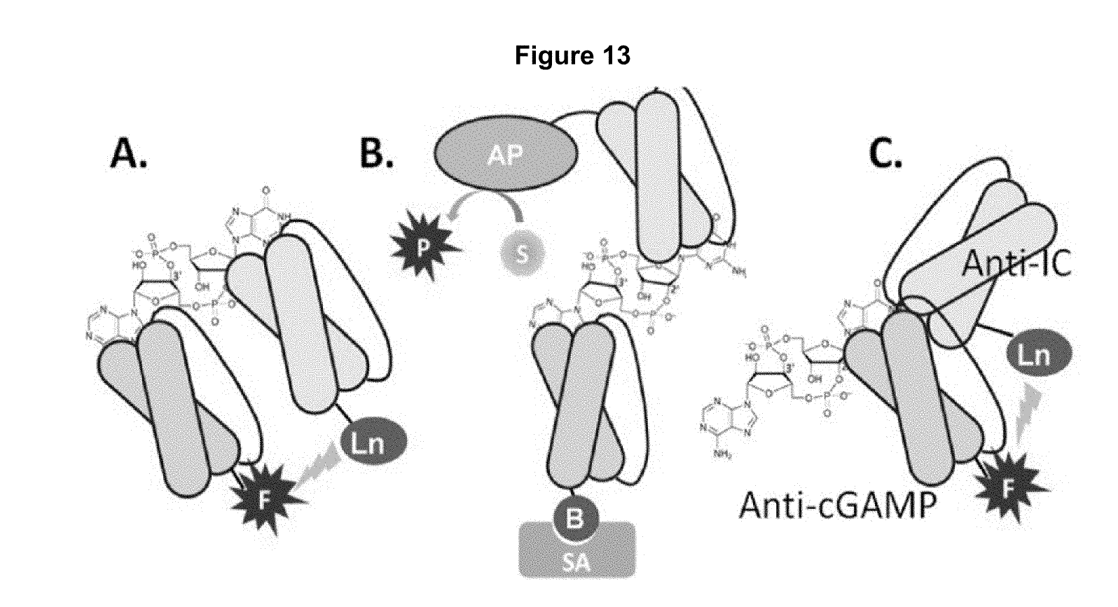

In some embodiments, cGAMP mAbs are generated using affinity maturation. In some embodiments, the mAb is an scFv. In some embodiments, scFvs to cGAMP having K_{d} of about 5 nM, or a K_{d} within the range of about 1 nM to about 5 nM, or a K_{d} of about 1 nM, are generated for a biomarker assay. In some embodiments, two scFvs to cGAMP having a simultaneous K_{d} of less than about 100 pM are generated for a cellular HTS assay. In some embodiments, a first scFv to cGAMP is generated, and a second scFv to the complex of the first scGv and cGAMP is generated (see, e.g., FIG. 13C). In certain such embodiments, the second scFv is generated using the same methods used to generate the first scFv, e.g., affinity maturation.

In some embodiments, assays for detection of cGAMP as a biomarker in cell and tissue extracts are developed. In some embodiments, assays for detection of cGAMP directly in cell lysates, e.g., for cellular HTS assays, are developed. In some embodiments, completive FP and/or TR-FRET immunoassays are developed for detection of cGAMP as a biomarker. In some embodiments, assays capable of detecting cGAMP in concentrations within the range of about 1 nM to about 100 nM are developed. In certain such embodiments, assays capable of detecting cGAMP in concentrations within the range of about 1 nM to about 100 nM, with a Z′ of at least 0.5 and/or a lower limit of detection (LLD) of less than about 0.5 nM are developed. In some embodiments, assays having less than +/−50% correlation between LC/MS and cGAS immunoassay results are generated. In some embodiments, competitive ELISA immunoassays are developed for detection of cGAMP as a biomarker.

In some embodiments, a cGAMP cellular HTS assay is validated using human cells. In some embodiments, cGAMP expression in cells from cGAMP+ and cGAMP− patients is evaluated by LC-MS. The cells from these patients are further analyzed via HTS assay in a blind fashion. In some embodiments, clinical information regarding the patient's medical history, number of classification criteria fulfilled, laboratory findings (including autoantibody specificities), and damage accrual data is obtained and measured using the Systemic Lupus International Collaborating Clinics/ACR Damage Index (SDI) (Gladman, et al., 1997, Arthritis Rheum 40(5):809-13).

In some embodiments, an S-TR-FRET detection method is utilized in a cGAMP cellular HTS assay. S-TR-FRET is a commonly used approach for homogenous HTS assays, e.g., assays for detection of phospho-proteins in cell extracts (Ayoub, et al., 2014, Front Endocrinol 5:94). Though it has not yet been used for small molecules, the recent examples of ELISAs for small molecules suggest that simultaneous binding of two antibodies to cGAMP is feasible. In some aspects, the potential gains in sensitivity and specificity over competitive FP or TR-FRET assay formats (Arola, et al., 2016, Anal Chem 88(4):2446-52; Arola, et al., 2017, Toxins 9(4):145), described above, combined with the advantages of a mix-and-read format make S-TR-FRET a highly desirable detection method for a cGAMP cellular HTS assay (FIG. 13A).

In some embodiments, assays capable of detecting endogenously produced cGAMP in cell extracts are produced. In some embodiments, assays capable of detecting endogenously produced cGAMP in cell extracts, with a Z′ of at least 0.5 and/or an LLD of less than about 10 pM are developed. In some embodiments, an ELISA, e.g., a sandwich ELISA, capable of detecting cGAMP at sub-picomolar sensitivity is produced (see, e.g., FIG. 13B). In certain such embodiments, the sandwich ELISA uses alkaline phosphatase (AP) as a reporter enzyme, in fusion with a secondary cGAMP scFv.

The invention will be further described in the following examples, which do not limit the scope of the invention described in the claims.

### Examples

The Examples that follow are illustrative of specific embodiments of the invention, and various uses thereof. They are set forth for explanatory purposes only, and are not to be taken as limiting the invention.

### Example 1: Development of FP Assay for cGAMP

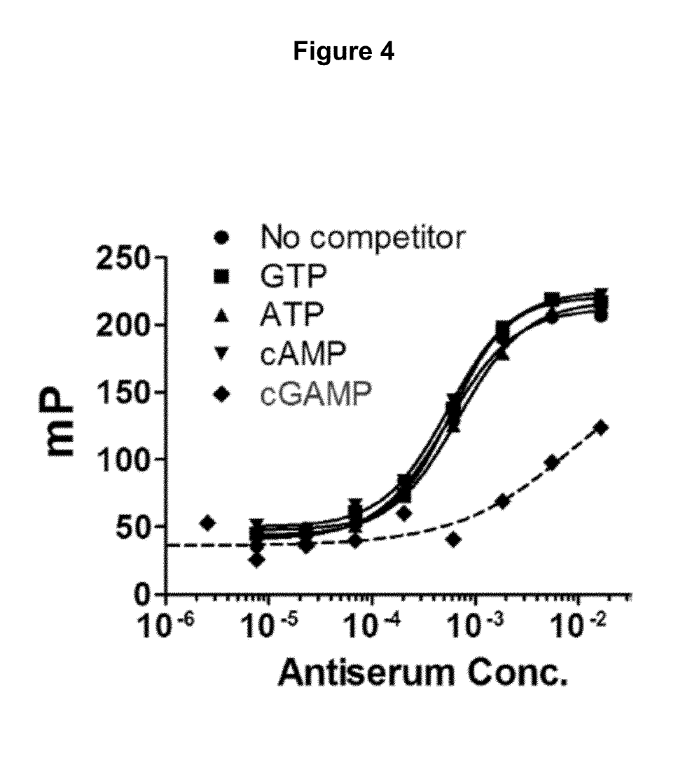

The development of immunogens and tracers (see FIG. 5) was carried out using reactive cGAMP analogs from BioLog (Bremen, Germany). A cGAMP immunogen was produced by conjugating an analog with an aminoethylthio linker at C-8 in the guanine ring (BioLog) to keyhole limpet hemocyanin (KLH) using standard EDC (1-ethyl-3-(3-dimethylaminopropyl) carbodiimide) chemistry; a BSA conjugate was also prepared for use in ELISAs. Monoclonal antibody production was performed by Harlan Sprague Dawley Inc. (Madison, Wis.). Of ten mice that were immunized, three showed strongly positive results in ELISAs. More significantly, antiserum from three mice produced a dose-dependent increase in the polarization of a fluorescein-cGAMP tracer (BioLog, also conjugated at C-8 of guanine), and this effect was competitively inhibited by unmodified cGAMP, but not by ATP or GTP; results from the mouse that was selected for monoclonal development are shown in FIG. 4. The polarization of the tracer increased with increasing cGAMP antiserum concentration, consistent with formation of an antibody-tracer complex. The binding curve was right-shifted by approximately two orders of magnitude when excess cGAMP (10 μM) was present, whereas ATP, GTP, and cAMP had no effect, indicating that the mAb/tracer interaction is specific. These results indicate that there is a high titer of antibody that binds the tracer and that the tracer is displaced by cGAMP, but not by the cGAS substrates ATP or GTP. In a continuing antibody development, 10 hybridoma clones were propagated and used for assay development. See Example 2.

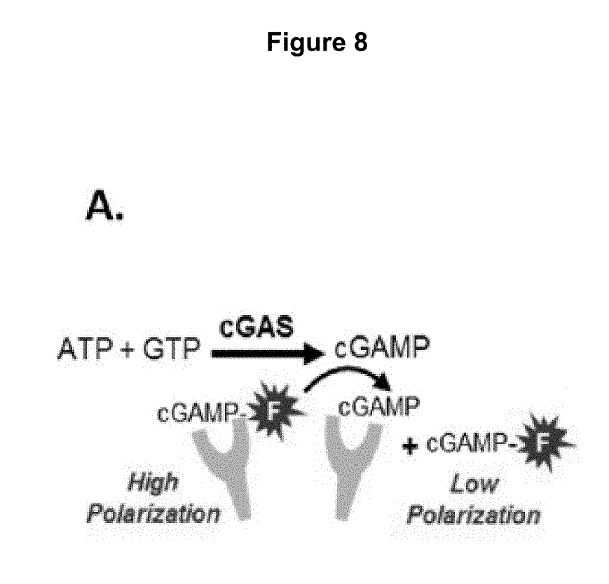

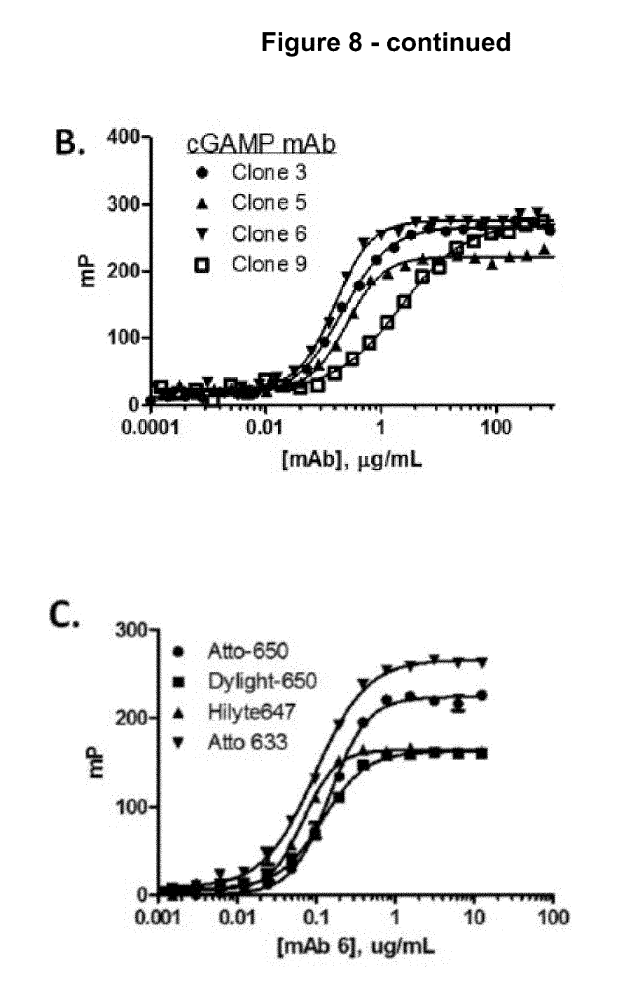

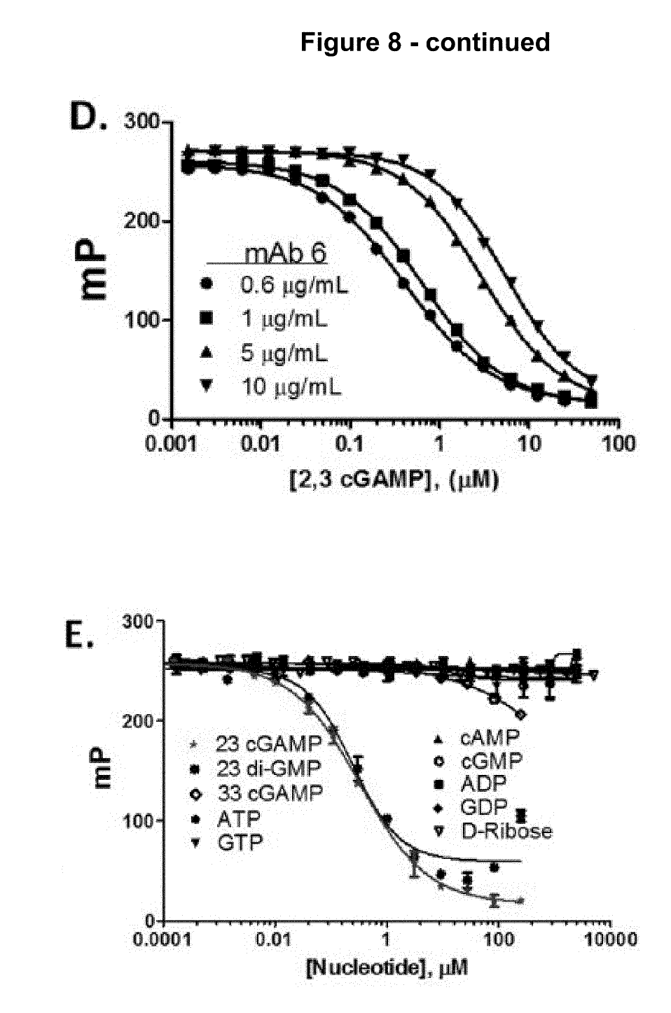

The commercially available tracer used has a fluorescein tag, which emits at 515 nM. Fluors that emit in the far red are much preferred for HTS, as background fluorescence from screening compounds is largely in the blue-to-green region of the spectrum (Vedvik, et al., 2004, Assay Drug Dev Technol. 2(2):193-203). Three cGAMP analogs with amino linkers to different positions (BioLog) were used to synthesize a collection of 15-20 tracers using amine reactive (NHS esters), far red fluors (e.g., Alexa Fluor, Dylight, and Atto dyes with emission above 600 nM), and purified by thin layer chromatography. Tracers were tested in a matrix fashion with mAbs using FP-based equilibrium binding and competition analysis to identify the pairs that yield the most sensitive and selective immunoassay; cGAMP mAb6 and the cGAMP-Atto 633 tracer (cGAMP linked to an Atto 633 fluor) were chosen for further assay development. Representative binding curves for the 10 cGAMP mAbs with cGAMP-Atto 633 and for cGAMP mAb6 with four different tracers are shown in FIGS. 8B and 8C. This type of iterative, empirical approach is the most effective for competitive immunoassay development, because minor changes in tracer structure can have large and unpredictable antibody-specific effects on both the sensitivity and the selectivity of a competitive immunoassay; attaching a fluor to an antigen at the same site used for the immunogen does not necessarily produce the best tracer.

### Signal

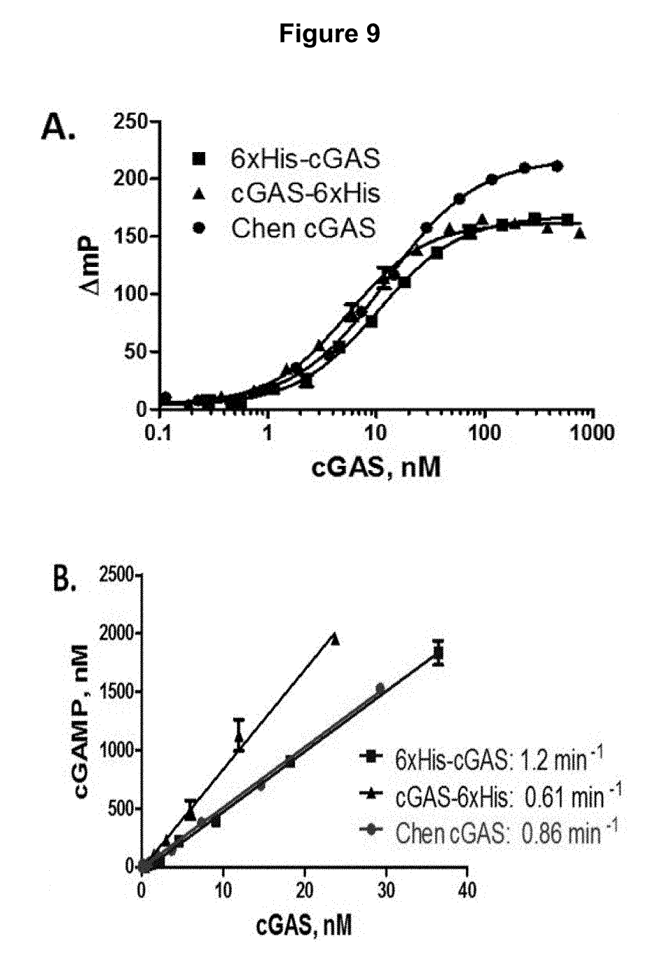

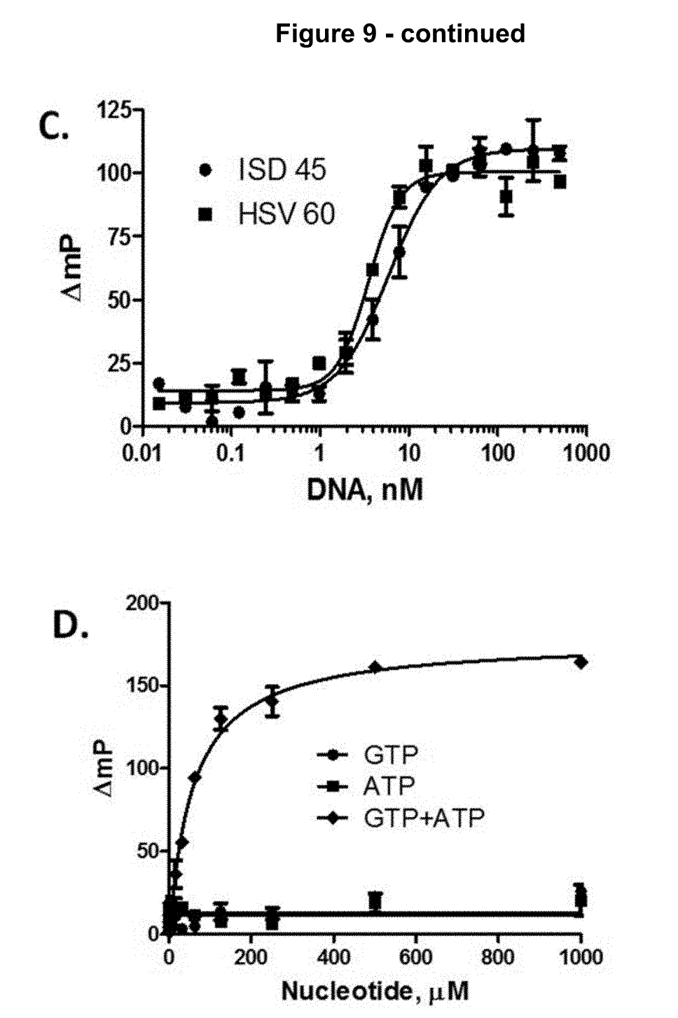

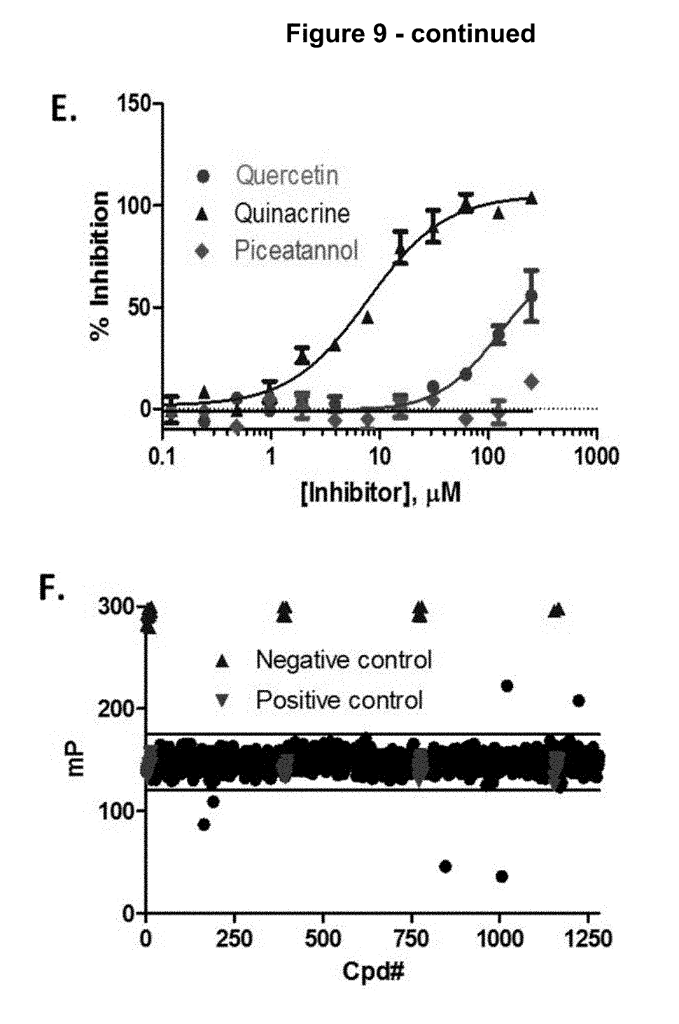

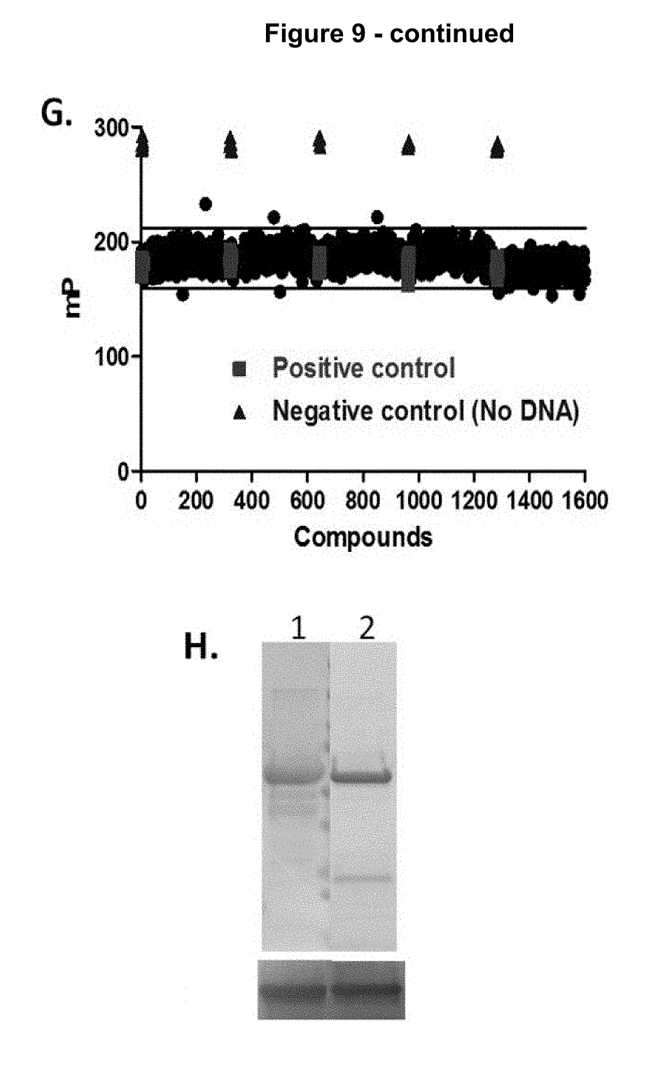

Though acceptable Z′ values can often be attained with much lower signals, a screening window of at least 80 mP is desirable for unambiguous identification of hits, and enzyme reactions should generally be adjusted to produce 80% of the maximum polarization shift; therefore the minimum goal is generally 100 mP. The maximum shift for the cGAMP assay was greater than 200 mP (FIG. 8D); an 80% decrease yielded a shift of more than 150 mP, which provides an outstanding screening window. These predictions are discussed in the cGAS enzyme studies described in Example 2, below (see also FIG. 9).

### Sensitivity

Enzymes are usually screened using the Kₘ concentrations of substrates to allow detection of competitive inhibitors, and assays are generally run under initial velocity conditions, i.e., less than 20% conversion of substrates to products. Therefore, enzyme assays need to be capable of generating a good signal at products levels of 5-10% of the substrate Kₘ. The Kₘ values for ATP and GTP were presumed to be at least 5 μM, given their millimolar concentrations in the cell. Accordingly, the target for practical cGAMP detection (i.e., a signal of at least 100 mP), was 500 nM. The sensitivity and dynamic range of the cGAMP assay could be tuned by changing the antibody concentration, which was present in excess over the tracer (FIG. 8D). At a concentration of 0.5-0.6 μg/ml mAb6, the half maximal signal occurred at 200-300 nM cGAMP, and a shift of approximately 130 mP was observed at 500 nM cGAMP (FIG. 8D); decreasing the antibody concentration further did not produce practical gains in sensitivity. The lower limit of detection (LLD), i.e., the lowest concentration that yields a positive Z′, was 12.5 nM.

### Selectivity

Use of the assay to measure cGAS enzyme activity requires an antibody that specifically binds cGAMP in the presence of an excess of substrates (ATP and GTP). As shown in FIG. 8E, there was no detectable binding of ATP and GTP to mAb6, even at more than 10⁴-fold excess over the half-maximal cGAMP concentration. Use of the assay for detection of cGAMP in the cell and/or tissue samples will require lack of cross reactivity with any other cellular nucleotides. Surprisingly and unexpectedly, the lack of binding of related nucleotides to mAb6, including cAMP, cGMP, ADP, and DGP indicates a very high level of specificity. Notably, these results indicate that mAb6 is highly specific for mixed 3′-5′, 2′-5′ phosphodiester bonds, and not for a base, as it recognized 2′,3′ cyclic di-GMP with essentially equal affinity to the antigen, 2′,3′ cGAMP (10₅₀ values of 250 and 330 nM, respectively), but it did not bind 3′,3′ cGAMP (see FIG. 8E). Importantly, neither of these other cyclic dinucleotides are known to be endogenous in mammalian cells.

### Example 2: Detection of cGAS

cGAS is one of four oligoadenylate synthases, nucleic acid sensors that activate innate immunity via production of short, 2′-5′ oligoadenylate secondary messengers (35); it catalyzes the formation of 2′,3′ cGAMP from ATP and GTP, with pyrophosphate as a byproduct. Binding of dsDNA and DNA:RNA hybrids to cGAS induces a conformational transition in an activation loop, not unlike the displacement of inhibitory domains by autophosphorylation in protein kinases (Zhang, et al., 2014, Cell Rep 6(3):421-30; Fabbro, et al., 2012, Methods Mol Biol 795:1-34). The full-length enzyme was used to insure that all of the potential allosteric sites were present and that the enzyme was able to sample its full conformational repertoire. Prior to running cGAS enzyme reactions, the effect of cGAS reaction components, including ATP and GTP Brij 35, NaCl, Brij 35, and dsDNA on the detection reagents was examined; no significant effects were observed. Assays using highly purified, full length human cGAS were performed. The full-length human cGAS was prepared according to Example 4, below. Additionally, third-party-provided cGAS (Sun, et al., 2013, Science 339(6121):786-91) was used for comparative purposes. Some enzyme assays were run in kinetic mode; i.e., the detection reagents were present during the enzyme reactions, and the plates were read periodically, and others were run in endpoint mode, using EDTA to quench the reaction.

Titration of the highly purified enzyme produced a sigmoidal dose-dependent polarization shift, as expected for a competitive binding assay, with a half-maximal change (EC₅₀) of 6-12 nM cGAS, depending on the preparation (FIG. 9A). The enzyme data was graphed as the net change in polarization; the absolute change produced by cGAMP was negative, as shown in FIG. 8. Conversion of polarization to cGAMP formation using a standard curve confirmed that product formation was linear with cGAS concentration (FIG. 9B); it was also linear with time for at least two hours, indicating that the enzyme was stable during the reaction period. There was essentially no quantitative activity data available for cGAS prior to the instant disclosure and the k_{cat} values of approximately 1 min⁻¹ determined herein (at non-saturating ATP and GTP) were the first reported cGAS kinetic parameters. The generation of a good signal (i.e., ≥100 mP) with less than 10 nM cGAS is important for two reasons. It reduces the time and cost to prepare for a large screen by minimizing enzyme usage; for example, screening 1M (one million) compounds at 10 nM cGAS will require only 12 mg of enzyme. It also enables accurate determination of inhibitor potencies into the nanomolar range without depletion of free inhibitor, which skews 10₅₀ measurements. Notably, the cGAS concentration may be further reduced after full optimization of the reaction for maximal activity.

The capability of the assay to detect activation of cGAS by dsDNA, a property critical to the physiological role of cGAS (Chen, et al., 2016, Nat Immunol 17(10):1142-9), was confirmed with a third-party-prepared enzyme, and with cGAS produced according to Example 4. Surprisingly and unexpectedly, the enzyme was shown to be highly sensitive to dsDNA, with a half maximal responses of 2.5 and 5.9 nM for herpes virus and Listeria sequences, respectively (FIG. 9D). It was also confirmed that cGAS requires both ATP and GTP for activity: the half maximal concentration of both nucleotides combined was 80 μM (FIG. 9D), thus substrate concentration in the cell is saturating. The potential for compound interference with the cGAMP FP and TR-FRET assays was tested using the 1280 LOPAC library of pharmacologically active compounds (Sigma), which includes many scaffolds found in larger screening libraries (FIG. 9F). Wells containing all cGAS enzyme reaction components except the cGAS enzyme; positive controls contained 1 μM cGAMP, negative controls contained no cGAMP. A pilot screen with 1600 diversity compounds (Life Chemicals) was performed (FIG. 9G). cGAS was used at 10 nM and reacted for 60 minutes; negative controls lacked dsDNA required for cGAS activation.

### Example 3: Development of TR-FRET Assay for cGAMP

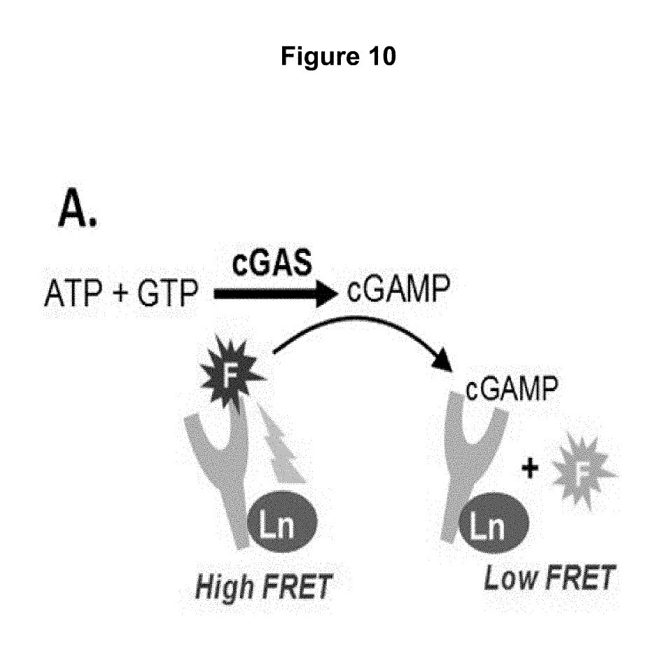

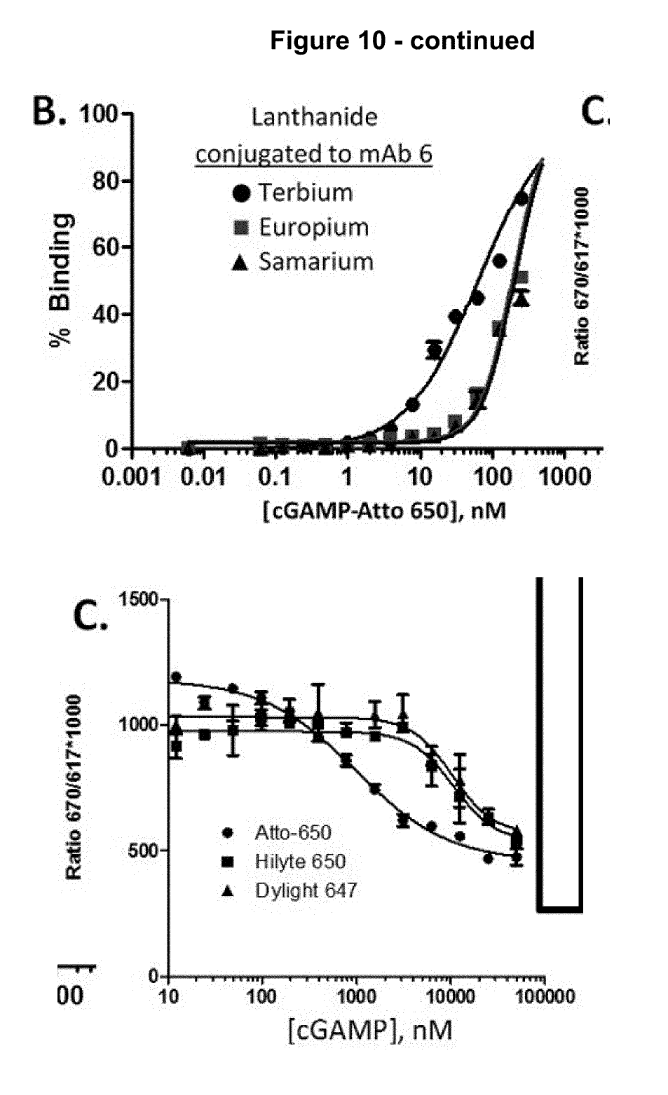

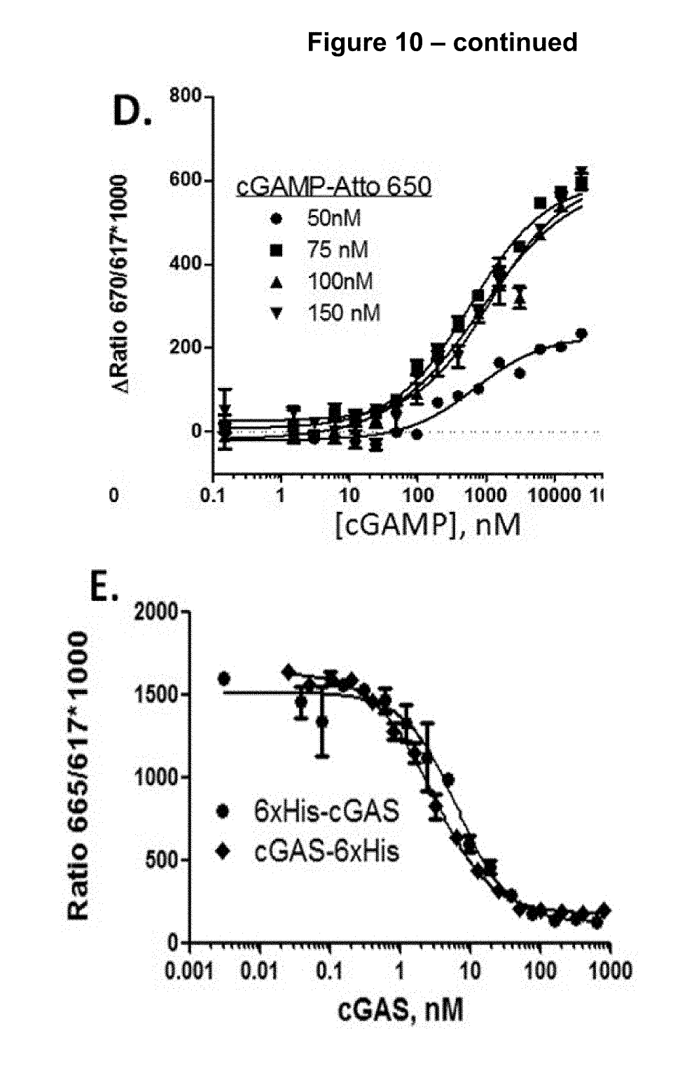

Having confirmed that cGAMP mAb6 has the required affinity and selectivity properties for a cGAS enzymatic assay in an FP format, development of the TR-FRET assay was relatively straightforward. Amine-reactive lanthanide chelates, including terbium, europium, and samarium, were conjugated to mAb6, and binding analysis was performed with a series of cGAMP-fluor tracers with overlapping excitation spectra. Notably, the TR-FRET assay differs from the FP assay in that the tracer, rather than the Ab, is present in excess; this minimizes consumption of expensive reagents. Additionally, the tracer fluors used for TR-FRET are generally more red-shifted than those of the FP assay in order to match the emission of lanthanides. The terbium-conjugated mAb 6 with the cGAMP-Atto 650 tracer resulted in the highest affinity of all the combinations tested, as exemplified in representative binding curves (FIGS. 10B and C). A tracer concentration of 75 nM provided the most sensitive detection (FIG. 10D), though the half maximal cGAMP concentration of 600 nM was approximately 2.5-fold higher than in the FP assay. Surprisingly and unexpectedly, cGAS enzyme was readily detected with the TR-FRET assay, with an EC₅₀ occurring at approximately 5 nM cGAS, similar to the FP assay (FIG. 10E). There was good concordance between the two assays, with respective cGAS-His K_{cat} values of 1.58 and 1.21 as determined by TR-FRET and FP, respectively. The Z′ value (n=16) for the Tr-FRET assay at 10 nM cGAS was 0.84.

### Example 4: Expression and Purification of Human cGAS

Though recombinant cGAS is commercially available, no associated activity data is available, and it is prohibitively expensive for HTS. Given the critical importance of a reliable source of high purity, functional cGAS for biochemical screening efforts, full-length human cGAS having a 6×His at either the N- or C-terminus were synthesized using a T7 expression vector and BL21 DE3 cells with eukaryotic tRNAs for rare codons (Rosetta DE3, Novagen). Following lysis, the soluble 6×His-cGAS and cGAS-6×His proteins were purified using immobilized metal ion affinity chromatography (IMAC); the 6×His-cGAS was further purified using cation exchange chromatography (HiTrap SP) on an Akta Start automated chromatograph system (GE Healthcare) with a 0.1-1M NaCl gradient. The two purified His-tagged cGAS constructs are shown in FIG. 9H; the purity of both was approximately 80% as determined by scanning the Coomassie blue-stained gel; identity was confirmed by western blot with an anti-cGAS antibody (Cell Signaling). The k_{cat} of both proteins, as measured with the cGAMP FP assay (see Example 2) was approximately 1.2 min⁻¹, which was similar to that obtained for full length, third-party-prepared cGAS.

Most cGAS structure/function studies have been performed with truncated constructs that include the primary DNA binding site and the catalytic domain, but lack the N-terminal 150-160 aa (Kato, et al., 2013, PloS One 8(10):e76983; Kranzusch, et al., 2013, Cell Rep 3(5):1362-8; Zhang, et al., Cell Rep 6(3):421-30). Using a cGAS produced according to this Example insures that all of the potential allosteric sites of cGAS were present, and that the enzyme was able to sample its full conformational repertoire. Notably, in this regard, a recent study showed that the N-terminal 160 aa domain makes an important contribution to DNA-dependent activation, both in vitro and in vivo (Tao, et al., 2017, J Immunol 198(9):3627-36).

### Example 5: Development of mAbs (scFvs) with Higher Affinity and Selective Epitope Recognition for Detection of cGAMP in Cells and Tissues

As described above, detection of endogenous cGAMP in biological samples requires greater sensitivity than that of the cGAS enzymatic assays of Examples 1-3 (see Table 3). To improve sensitivity, three antigens that are structurally distinct from the guanine C-8-linked immunogen of Examples 1-3 are be produced (FIG. 11). These antigens may lead to a higher affinity monoclonal, and will allow development of a pair of antibodies that recognize different epitopes, enabling a sandwich based assay (as in the tacrolimus ELISA; Wei T Q, et al., 2014, Clin Chem 60(4):621-30).

TABLE 3
cGAMP immunoassay development summary

| | Detection | Assay | Signaling |
|---|---|---|---|
| Application | Range | Format | Mechanism |
| Enzymatic HTS assay | 50 nM-50 μM | Competitive | FP, TR-FRET |
| Biomarker assay | 1 nm-100 nM | Competitive | FP, TR-FRET |
| Cellular HTS assay | 20 pM-2 nM | Sandwich | S-TR-FRET |

Generation of Additional mAbs to cGAMP Using Structurally Distinct Antigens

cGAMP is conjugated to KLH via 6-aminohexyl carbamoyl linkers to the ribose 3′-hydroxy group of the guanosine (Biolog C191), to the ribose 3′-hydroxy group of adenosine (Biolog C192), and via a 2-4 carbon linker to the N6 of the adenine ring using EDC chemistry to generate antigens with three epitope presentations distinct from the guanine C8-linked cGAMP antigen of Examples 1-3. The ribose-linked cGAMP analogs are available from Biolog, and the adenine N6-linked analog is provided as a custom synthesis. The latter analog is important because it will present cGAMP in the opposite orientation relative the antigen of Examples 1-3, i.e., with the guanine ring fully exposed and the adenine ring less accessible, because there is precedent for such antibodies that can discriminate with more than 100-fold selectivity between cGMP and cAMP (Wescott, et al., 1985, J Cyclic Nucleotide Protein Phosphor Res 10(2):189-96). Moreover, the site attachment and conjugation chemistry used for antigen synthesis can have a profound effect on affinity and selectivity, as observed through efforts to develop anti-nucleotide antibodies for ADP, GDP, AMP, etc. (Kleman-Leyer, et al., 2009, Assay Drug Dev Technol 7(1):56-67).

As in Example 1, monoclonal antibody production is performed by Envigo (formerly Harlan; Madison, Wis.) using ten mice for each antigen. Mice are chosen for hybridoma development based on analysis of antiserum (tail bleeds) using competitive ELISAs and FP-based competition assays. Hybridomas are screened using the cognate antigen, as well as the two non-cognate antigens to identify mAbs that may exhibit some differences in epitope recognition. The most promising mAbs are tested using FP-based equilibrium binding and competition assays with a panel of tracers made by conjugating to the four different positions on cGAMP (FIG. 11). Relative binding affinities to the cognate vs. non-cognate tracers will be used to assess differences in epitope recognition; a standard sandwich ELISA assay will be used to test for simultaneous binding. Given the success in developing sandwich ELISAs for tacrolimus, an immunosuppressive drug, and imantibin, a cancer drug (Saita, et al., 2017, Anal Chim Acta 969:72-8; Wei, et al., 2014, Clin Chem 60(4):621-30), it is not unreasonable to expect that one or more pairs of antibodies may be identified that bind simultaneously. If not, competitions studies with cyclic dinucleotide variants such as 2′,3′-cyclic di-AMP and 2′-3′-cyclic di-GMP (Biolog) are performed to obtain more information on epitope recognition properties, and to select one or two antibody pairs with the best combination of affinity, specificity, and differential epitope recognition for in vitro evolution.

Affinity Maturation of cGAMP mAbs

Affinity maturation is performed using PCR-based mutagenesis of scFvs combined with FACS-based enrichment of yeast-displayed clones, an approach that can yield gains in affinity of more than 1000-fold (including scFvs for fluorescein and, more recently, the lanthanide chelate DOTA, with respective K_{d}s of 0.27 and 8.2 pM; Table 3; Boder, et al., 2009, Proc Nat Acad Sci USA 97(20):10701-5; Orcutt, et al., 2011, Nucl Med Biol 38(2):223-33). Unlike ribosome or bacterial display, yeast display allows the use of fluorescence-activated cell sorting (FACS) for quantitative and exhaustive screening of large populations to optimize antigen binding affinity and kinetics (Boder, et al., 2012, Arch Biochem Biophys 526(2):99-106).

Generally, established FACS methods are performed, with minor modifications to reduce the mutational bias of error-prone PCR (Orcutt, et al., 2011, Nucl Med Biol 38(2):223-33), and to increase selection of stable scFvs (Julian, et al., 2017, Sci Rep 7:45259). scFvs are cloned using RT-PCR of RNA prepared from selected cGAMP-mAb hybridoma cells (3-4 clones) using standard methodology to link the V_{H} and V_{L} domains with a Gly-Ser linker and inserted into the pCTCON-T yeast shuttle vector in fusion with the adhesion subunit of the yeast agglutinin protein Aga2p for surface display. pCTCON-T includes a Gall promoter for inducible expression in yeast and a C-terminal 6×His tag for affinity purification (FIGS. 12A and 12B). Following characterization of the native cGAMP scFvs for binding affinity and kinetics using FP-based binding assays with cGAMP tracers (notably, the much smaller size of scFv domains-28 kDa vs. 150 kDa—may facilitate simultaneous antibody binding), each is subjected to multiple (5-10) rounds of directed evolution by random mutagenesis using PCR conditions optimized to produce 1-9 amino acid mutations per scFv gene (Chao, et al., 2006, Nat Protoc 1(2):755-68). Two types of thermostable polymerases are used at each round to minimize the mutational bias of error-prone PCR (Orcutt, et al., 2011, Nucl Med Biol 38(2):223-33). Though targeted mutagenesis of CDRs is often used, the entire variable domains (V_{H} and V_{L}) are subjected to mutations because a) most affinity-enhancing mutations occur outside the primary binding interface, either at the periphery or outside of the CDRs, and b) compensatory mutations in the framework region can counteract the destabilizing effects of affinity-enhancing mutations in the CDRs (Julian, et al., 2017, Sci Rep 7:45259).

The mutant scFv libraries are amplified to produce a quantity sufficient for yeast transformation and are cloned into pCTCON-T by homologous recombination in yeast (Oldenburg, et al., 1997, Nucleic Acids Res. 25(2):451-2). As the PCR insertion products are also homologous to each other, additional recombination events occur between inserts and lead to greater library diversity. Each of the cGAMP scFv-expressing yeast libraries is sorted by FACS for improved binders (2-3 times at least selection round), using at least 5 times the estimated library diversity. cGAMP-Atto 633 tracers (cGAMP linked to an Atto 633 fluor) with the cognate linker attachment site (FIG. 11) are used as labels to sort scFvs for increased affinity, and for desired epitope recognition properties. For example, an scFv derived from a mAb that was raised to a KLH-linker-N6-adenine-cGAMP antigen will be sorted using an Atto 633-linker-N6-adenine-cGAMP label. Simultaneously, labeling with fluorescein-conjugated Protein A (FI-ProtA) is used to sort for high expression of scFvs. Affinity-enhancing mutations can be destabilizing, and protein A only binds to properly folded scFvs; thus it is a more stringent probe than one that detects a small domain on the scFv (such as a Myc-tag) (Julian, et al., 2017, Sci Rep 7:45259). cGAMP-Atto 633 is used at a concentration of approximately ½-⅓ of the average K_{d} of the previous library (in early rounds) or at 2×K_{d} followed by displacement by unlabeled cGAMP for 2-3 dissociation half-times (in later rounds). Yeast expressing the best 0.01-0.1% of binders are collected.

When an affinity of 1-5 nM is attained with any of the libraries, individual scFvs are cloned and characterized for affinity and selectivity for development as a biomarker assay (see Example 6, below). Further rounds are directed toward evolving scFv pairs with picomolar affinity that can bind cGAMP simultaneously for an S-TY-FRET assay (see Example 7, below). Tracers with the non-cognate structure and a non-overlapping fluor (e.g., Alexa Fluor 405) are used for counter-screening in latter rounds to remove scFvs that recognize the same epitope as another pair member, e.g., adenine specific vs. guanine specific binders.

### Example 6: Development of Competitive FP and/or TR-FRET Immunoassays for Detection of cGAMP as a Biomarker

### Reagent Development

A competitive immunoassay format with FP and/or TR-FRET signals is used for biomarker assays, because such formats are the simplest configurations for the desired detection range, and because these formats are widely used. For example, widely used assays for cyclic mononucleotides with practical detection of less than 5 nM use competitive FP and TR-FRET formats (e.g., Lance Ultra cAMP, Perkin-Elmer (Norskov-Lauritsen, et al., 2014, Int J Mol Sci 15(2):2554-72)). Assay development is similar to that described in Example 1 (see FIG. 5). Three or four scFVs generated from affinity maturation are selected based on affinity (1 nm≤K_{d}≤5 nM) and specificity (negligible cross-reactivity with other endogenous nucleotides) and tested in a matrix fashion with tracers using FP. Selection is performed from an early round of affinity maturation, as scFVs with a K_{d} lower than 1 nM would not be as useful for such an assay configuration. Additionally, the scFVs may be modified to optimize their utility for FP-based detection. FP assays are based on the rotational mobility of the fluor, which is in turn proportional to its effective molecular volume. Though there are other contributing factors (e.g., the length and flexibility of linkers used to synthesize tracers), the difference in size between the free and bound states of the tracer has the greatest effect on the magnitude of the assay signal. The smaller size of the scFv fragments relative to full-size IgGs may result in a smaller in tracer polarization upon binding, thus decreasing the assay window. If this is the case, fusions to the C-terminus of a heavy chain, such as glutathione transferase (27 kDA) or maltose binding protein (42 kDA), are produced and tested. Following identification of the most promising scFv/tracer pairs using FP binding analyses, the scFvs are labeled with lanthanide chelates and tested with tracers with overlapping spectra for development of TR-FRET assays. The FP and TR-FRET assays are fully characterized for sensitivity, specificity, signal variability/dynamic range (Z′), and potential interferents, as described in Examples 1-3, above.

### Assay Validation

The CGAMP biomarker assay is validated by direct comparison with LC/MS detection using a variety of therapeutically relevant cell and tissue samples from animals and humans. cGAMP levels in cell and tissue extractions are quantified by LC/MS as described below, and compared to FP and/or TR-FRET assay measurements.

cGAMP from Monocytes

THP-1 cells (0.2, 1, 5, 25M) are transfected with herring testis DNA (0.1, 0.5, 2.5, 12.5 μg) with Lipofectamine 2000. After 4 hours, cGAMP is isolated from THP-1 cells alone or THP-1 cells transfected with double-stranded DNA (dsDNA) by a methanol extraction procedure.

THP1 cells are lysed with 1 ml of 80% methanol spiked with 5 nM heavy isotope-labeled cGAMP (cGAMP*) containing ¹³C-, ¹⁵N-labeled AMP as an internal standard. Following sonication and harvesting, cGAMP is further purified with a solid-phase extraction column (Oasis WAX column; Waters) and resuspended in 50 μl of Optima LC-MS water (Thermo Scientific) for mass spectrometry. The cGAMP concentration (estimated 1˜100 nM) is measured by LC-MS, FP, and/or TR-FRET immunoassays for comparison. The mass spectrum peak area of the endogenous cGAMP and internal standard (100 nM final concentration) is quantified by QuanLynx software (Waters). The ratio of the peak area from endogenous cGAMP and internal standard is used to determine the concentration of endogenous cGAMP

cGAMP from Diseases Cells and Tissue

### Trex1−/− Mice

The dominant clinical manifestation in Trex1−/− mice is an autoimmune myocarditis (Morita, et al., 2004, Mol Cell Biol 24(15):6719-27). The profound reduction or loss of disease manifestations and extended survival observed in Trex1−/− mice deficient in either STING or cGAS (Gao, et al., 2015, Proc Natl Acad Sci USA 112(42):E5699-705; Gray, et al., 2015, J Immunol 195(5):1939-43), indicate that IFN-I is induced through the cGAS-STING pathway. cGAMP is detectable in the high-pM or low-nM range in the hearts of Trex1−/−, but not Trex1−/−x cGAs−/− mice (Gao, et al., 2015, Proc Natl Acad Sci USA 112(42):E5699-705). This observation was confirmed by LC-MS. At 3 months of age, Trex1−/− and Trex1+/− littermate controls are scarified (n=10 mice each) to obtain the hearts. After taking small slivers for RNA and histology, the hearts are bisected; one half is tested for cGAMP by LC-MS, and the other by competitive FP and/or TR-FRET immunoassays. Quantification by mass spectrometry is achieved using heavy isotope-labeled cGAMP (cGAMP*) containing ¹³C-, ¹⁵N-labeled AMP as internal standard, as in the cGAMP measurement in THP1 cells, described above.

### Example 7: Development of S-TR-FRET Format as a Cellular HTS Assay for cGAMP Modulators

As described above, the potential gains in sensitivity and specificity over competitive FP or TR-FRET assay formats (Arola, et al., 2016, Anal Chem 88(4):2446-52; Arola, et al., 2017, Toxins 9(4):145), as well as the advantages of a mix-and-read format, make S-TR-FERT (FIG. 13) a highly desirable detection method for a cGAMP cellular HTS assay. The critical aspect of S-TR-FRET assay development is the generation of a pair of scFvs with the required affinity and epitope recognition properties (see Example 5). The desired properties of a candidate scFv, if indicated by FACS, are confirmed using FP-based assays with cognate and non-cognate tracers. More detailed characterization of scFv affinity and specificity is carried out by tested in matric fashion in an S-TR-FRET format—the FP assay format is not sufficiently sensitive to allow measurement of binding affinities in the picomolar range. For initial screening of pairs, terbium and Atto-650 are used as the donor and acceptor, respectively, because the pair provides the best combination of sensitivity and signal:background ratio in assay development projects. All candidate scFvs are conjugated with an amine-reactive terbium chelate (LanthaScreen, ThermoFisher) and the Atto-650 fluor (Sigma), and tested in matrix fashion for binding in the presence of 1 nM cGAMP. Many clones can be tested with such an approach, as the lanthanide and fluor conjugations are simple, one-step reactions, and the unreacted labels can be removed using gel-filtration spin columns. The most promising pairs (2-3) are used for further assay development, including testing of different lanthanide and fluor pairs, and optimization of conjugation reactions. The final assay is fully characterized for sensitivity, dynamic range, selectivity, and tolerance of common reagents such as salts, metal chelators, reducing agents, and detergents, as in Examples 1-3.

### Assay Validation

While most cGAS/STING pathway studies have utilized the human monocyte cell lines THP-1, pDCs are clearly the primary cell type responsible for tumor surveillance and initiation of an IFN-driven innate immune response (Bode, et al., 2016, Eur J Immunol 46(7):1615-21; Corrales, et al., 2017, Cell Res 27(1):96-108). Therefore, in addition to THP-1 cells, human pDC line Cal-1 cells (Maeda, et al., 2005, Int J Hematol 81(2):148-54), recently shown to have a functional cGAS/STING pathway, including a robust cGAS-dependent production of IFNβ in response to cytosolic DNA (Bode, et al., 2016, Eur J Immunol 46(7):1615-21), are used for validation of the S-TR-FRET assay. Cells are grown to high density in 384-well plates and transfected with herring testis DNA (0.1, 0.5, 2.5, 12.5 μg) with Lipofectamine 2000. Following media removal, cells are lysed using buffer with a non-ionic detergent and other components that are compatible with S-TR-FRET reagents. S-TR-FRET reagents will be added, and plates are read as in the TR-FRET assay of Example 3. Unstimulated controls and spiked samples are used for comparison. A standard curve in lysis buffer is used to convert TR-FRET signals to quantitative cGAMP measurements.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

1. An antibody that specifically binds G(2′-5′)pA(3′-5′)p (cGAMP).

2. The antibody of claim 1, wherein the antibody specifically binds cGAMP in the presence of excess ATP, GTP, or both.

3. (canceled)

4. The antibody of claim 1, wherein the antibody binds cGAMP with a K_{d} of less than 100 nM, less than 5 nM, or less than 100 pM.

5. The antibody of claim 1, wherein the binding is in a biological sample.

6. The antibody of claim 5, wherein the biological sample is a cell extract, a tissue extract, and/or a cell lysate.

7. The antibody of claim 1, wherein the antibody is:
(a) a mouse monoclonal antibody;
(b) a single-chain variable fragment (scFv); and/or
(c) conjugated to a Tb-chelate or an Eu-chelate.

8-9. (canceled)

10. An assay method for measuring cGAMP produced in an enzymatically catalyzed reaction, comprising:
(a) contacting a biological sample with the antibody of claim 1;
(b) measuring a signal; and
(c) detecting cGAMP in the sample;
thereby measuring cGAMP produced in the reaction catalyzed by cyclic GMP-AMP synthase (cGAS).

11. (canceled)

12. An assay method for measuring cyclic GMP-AMP synthase (cGAS) activity, comprising:
(a) contacting a biological sample with the antibody of claim 1;
(b) measuring a signal; and
(c) detecting cGAMP in the sample;
thereby measuring cyclic GM P-AMP synthase (cGAS) activity.

13. An assay method for measuring cGAMP as a biomarker, comprising:
(a) contacting a biological sample with the antibody of claim 1;
(b) measuring a signal; and
(c) detecting cGAMP in the sample;
thereby detecting the biomarker.

14-17. (canceled)

18. An assay kit for detecting and measuring cGAMP produced in an enzymatically catalyzed reaction, comprising the antibody of claim 1.

19. An antibody pair comprising a first antibody and a second antibody, wherein:
the first antibody specifically binds G(2′-5′)pA(3′-5′)p (cGAMP) in the presence of excess ATP and GTP; and
the second antibody specifically binds:
(a) cGAMP, simultaneously with the first antibody; or
(b) a complex of the first antibody and cGAMP.

20. The antibody pair of claim 19, wherein the first antibody binds cGAMP with a K_{d} of less than 100 nM, less than 5 nM, or less than 100 pM; and the second antibody binds cGAMP or a complex of the first antibody and cGAMP with a K_{d} of less than 100 nM, less than 5 nM, or less than 100 pM.

21. The antibody pair of claim 19, wherein the first antibody and/or the second antibody comprise a single-chain variable fragment (scFv).

22. The antibody pair of claim 19, wherein the binding is in a biological sample;
wherein the biological sample is a cell extract, a tissue extract, and/or a cell lysate.

23-24. (canceled)

25. The antibody pair of any of claim 19, wherein:
(a) the first antibody is conjugated to a Tb-chelate or an Eu-chelate and the second antibody is conjugated to a fluorescent label; or
(b) the first antibody is conjugated to a fluorescent label; and the second antibody is conjugated to a Tb-chelate or an Eu-chelate.

26-27. (canceled)

28. An assay method for measuring cGAMP produced in an enzymatically catalyzed reaction, comprising:
(a) contacting a biological sample with the antibody pair of claim 19;
(b) measuring a signal; and
(c) detecting cGAMP in the sample;
thereby measuring cGAMP produced in the reaction catalyzed by cyclic GMP-AMP synthase (cGAS).

29. (canceled)

30. An assay method for measuring cyclic GMP-AMP synthase (cGAS) activity, comprising:
(a) contacting a biological sample with the antibody pair of claim 19;
(b) measuring a signal; and
(c) detecting cGAMP in the sample;
thereby measuring cyclic GMP-AMP synthase (cGAS) activity.

31. An assay method for measuring cGAMP as a biomarker, comprising:
(a) contacting a biological sample with the antibody pair of claim 19;
(b) measuring a signal; and
(c) detecting cGAMP in the sample;
thereby detecting the biomarker.

32. (canceled)

33. An assay kit for detecting and measuring cGAMP produced in an enzymatically catalyzed reaction, comprising the antibody pair of claim 19.
